(19) United States
(12) Patent Application Publication
(10) Pub. No.: US 2004/0253250 A1
(43) Pub. Date: Dec. 16, 2004

(54) Compositions and methods for regulating lymphocyte activation

(75) Inventors: Jeffrey A. Ledbetter, Shoreline, WA (US); Martha Hayden-Ledbetter, Shoreline, WA (US); William A. Brady, Bothell, WA (US); Laura S. Grosmaire, Hobart, WA (US); Che-Leung Law, Shoreline, WA (US); Raj Dua, Issaquah, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: XCYTE Therapies, Inc., Seattle, WA

(21) Appl. No.: 10/646,381

(22) Filed: Aug. 21, 2003

Related U.S. Application Data
(63) Continuation of application No. 09/252,150, filed on Feb. 18, 1999, now abandoned.
(60) Provisional application No. 60/108,683, filed on Nov. 16, 1998.
(60) Provisional application No. 60/075,274, filed on Feb. 19, 1998.

(51) Int. Cl. A61K 39/00

(52) U.S. Cl. 424/185.1; 424/93.71

(57) ABSTRACT

The present invention relates to regulation of lymphocyte activation. In particular, it relates to compositions and methods for regulating lymphocyte activation by selectively binding multiple cell surface antigens expressed by the same lymphocyte.

### 1. INTRODUCTION

The present invention relates to regulation of lymphocyte activation. In particular, it relates to compositions and methods for regulating lymphocyte activation by selectively binding multiple cell surface antigens expressed by the same lymphocyte. Antigen aggregation can be achieved in vitro by incubating lymphocytes with immobilized ligands or antibodies or antibody fragments specific for the target antigens. In addition, multi specific molecules that contain multiple binding specificities in a single soluble molecule are particularly useful in aggregating multiple antigens in vivo resulting in lymphocyte activation. Multispecific molecules may also be constructed to inhibit lymphocyte activation by blocking the delivery of activation signals to the cells. Therefore, the invention is useful in regulating T and B cell immune responses in vitro and in vivo.

### 2. BACKGROUND OF THE INVENTION

2.1. T Cell Receptor/CD3 Complex

Mature T lymphocytes (T cells) recognize antigens by the T cell antigen receptor (TCR) complex. In general, each TCR/CD3 complex consists of six subunits including the clonotypic disulfide-linked TCRα/β or TCRγ/δ heterodimers and the invariant CD3 complex (M. M. Davis, Annu. Rev. Biochem., 59: 475, A. C. Chan et al., Annu. Rev. Immunol., 10: 555). The TCR α, β, γ, and δ chains are 40 to 50 kDa glycoproteins encoded by T cell specific genes that contain antibody-like variable (V), joining (J), and constant (C) regions (S. M. Hedrick et al., Nature, 308: 149; S. M. Hedrick et al., Nature, 308: 153). The TCR heterodimers are the antigen binding subunits and they determine the specificity of individual T cells. α/β heteroexpressing cells constitute more than 90% of peripheral T cells in both humans and mice, and they are responsible for the classical helper or cytotoxic T cell responses (M. M. Davis, Annu. Rev. Biochem., 59: 475; A. C. Chan et al., Annu. Rev. Immunol., 10: 555). In most cases, TCRα/β ligands are peptide antigens presented by the major histocompatibility complex (MHC) Class I or Class II molecules. In contrast, the nature of TCRγ/δ ligands is not as well defined, and may not involve presentation by the MHC proteins (Y.-H. Chien et al., Annu. Rev. Immunol., 15: 511).

The invariant CD3 complex is made up of four relatively small polypeptides, CD3δ (20 kDa), CD3ε (20 kDa), CD3γ (25 kDa) and CD3ζ (16 kDa). CD3δ, ε, and γ chains show a significant degree of similarity to each other in their amino acid sequences. They are members of the immunoglobulin (Ig) supergene family, each of them possesses a single extracellular Ig-like domain. In contrast, CD3ζ only has a nine amino acid extracellular domain and a longer cytoplasmic domain when compared to CD3δ, ε, and γ. The cytoplasmic domains of the CD3 chains contain one to three copies of a conserved motif termed an immunoreceptor tyrosine-based activation motif (ITAM) that can mediate cellular activation. One consequence of TCR/CD3 complex ligation by peptide-MHC ligands is the recruitment of a variety of signaling factors to the ITAMs of the CD3 chains. This initiates the activation of multiple signal transduction pathways, eventually resulting in gene expression, cellular proliferation and generation of effector T cell functions (A. Weiss and D. R. Littman, Cell, 76: 263; R. Wange and L. E. Samelson, Immunity, 5: 197).

The assembly and expression of the TCR complex are complex and tightly regulated processes; exactly how different chains of the receptor complex contribute to these remain to be fully elucidated. Nevertheless, it is well established that surface expression of a TCR complex requires the presence of TCRα/β or TCRγ/δ plus CD3δ CD3ε, CD3γ, and CD3ζ chains (Y. Minami et al., Proc. Natl. Acad. Sci. USA., 84: 2688; B. Alaracon et al., J. Biol. Chem., 263: 2953). Absence of any one chain renders the complex trapped in the cytoplasm and subjects them to rapid proteolytic degradation (C. Chen et al., J. Cell Biol. 107: 2149; J. s. Bonifacino et al., J. Cell Biol. 109: 73). The precise stoichiometry of a TCR/CD3 complex is unknown. Several lines of evidence have suggested that one TCR/CD3 complex may contain two copies of the TCR heterodimer, a CD3ε/δ heterodimer, a CD3ε/γ heterodimer and a CD3ζζ homodimer to constitute a decameric complex (R. S. Blumberg et al., Proc. Natl. Acad. Sci. USA., 87: 7220; M. Exley et al., Mol. Immunol., 32: 829). In this complex, the TCR heterodimers and CD3ζ homodimers are covalently linked by disulfide bonds, while the CD3ε/δ and CD3ε/γ heterodimers are not covalently linked. Furthermore, the interaction among CD3ε/δ, CD3ε/γ, CD3ζζ, and TCRα/β or TCRγ/δ chains has been shown to be non-covalent.

Assembly of the TCR/CD3 complex begins with pairwise interactions between individual TCRα, TCRβ chains with the CD3 chains in the endoplastmic reticulum (ER) leading to the formation of intermediates consisting of a single TCR chain in association with the CD3 chains (B. Alarcon et al., J. Biol. Chem., 263: 2953; N. Manolios et al., EMBO J., 10: 1643). Transfection studies conducted in non-lymphoid cells shows that TCRα can associate with CD3δ and CD3ε but not CD3ζ whereas TCRβ can associate with CD3δ, ε, and γ but no CD3ζ (N. Manolios et al., EMBO J., 10: 1643; T. Wileman et al., J. Cell Biol., 122: 67). The incorporation of the CD3ζ chain appears to be the rate-limiting step for the formation of a mature TCR/CD3 complex. TCRα/β, CD3δ, ε, and γ chains are strictly required to be present in the ER before CD3ζ can assemble with the partial TCR/CD3 complex to form the final product for surface expression (Y. Minami et al., Proc. Natl. Acad. Sci. USA., 84: 26880. Association between the TCR and CD3 chains seems to depend largely on the charged amino acid residues in their transmembrane domains. Positively charged amino acid residues are present in the transmembrane domains of the TCRα/β chains, an arginine and a lysine for TCRα and a lysine for TCRβ. Negatively charged amino acids are found in the transmembrane domains of the CD3 chains, a glutamic acid for CD3γ and an aspartic acid for each of CD3ε, δ and ζ. Formation of salt bridges due to these charged amino acid is believed to be the main force driving the association between the TCRα/β chains and the CD3 chains (C. Hall et al., Int. Immunol., 3:359; P. Cosson et al., Nature, 351:414). A model for a mature TCR/CD3 complex compatible to the above transfection and biochemistry data has been proposed. In this model, one copy each of CD3ε/δ, CD3 ε/γ and CD3ζ/ζ form the core of the receptor complex with two copies of TCRα/β on the outside. TCRα and TCRβ chains may pair with CD3δ, ε or γ. The disulfide-linked CD3ζζ may preferentially pair with TCRα due to the additional negatively charged amino acid in the transmembrane domain of TCRα.

Although the assembly and expression of the TCR/CD3 complex have been extensively studies, relatively little is known about the potential functions of the extracellular domains of the CD3δ, ε or γ chains. Recent studies on the crystal structure of a TCR-anti-TCR complex has provided evidence for the presence of a binding pocket in the TCRβ chain large enough to accommodate the extracellular domain of CD3ε (J.-H. Wang et al., EMBO J., 17: 10; Y. Ghendler et al., J. Exp. Med., 187:1529). On the other hand, using deletional analysis a region proximal to the transmembrane domains of the CD3δ, ε or γ chains with a conserved Cys-X-X-Cys motif has been implicated to mediate CD3 chain hetero-dimerization (A. Borroto et al., J. Biol. Chem., 273: 12807). Members of the Ig supergene family are well known for their functions as adhesion molecules. Therefore it is not surprising that ligands may exist for the extracellular domains of CD3 of Ig-like domains. Accordingly, the interaction between CD3 chains and their potential ligands may play crucial roles in regulating T lymphocyte activation.

The absence of a system to produce soluble CD3 complexes in their native conformations is one underscoring reason for a lag of information on functions of the extracellular domains of the CD3 chains. Numerous monoclonal antibodies (mAbs) have been raised against the TCR/CD3 complex; many of them specifically recognize the CD3 complex. Moreover, the reactivity of most anti-CD3 mAbs falls into two categories: anti-CD3 mAbs that can recognize the CD3ε chain alone and anti-CD3 mAbs that only recognize a conformation epitope believed to be generated by a native interaction between the CD3ε chain and either the CD3δ or CD3γ chain (A. Salmeron et al., J. Immunol., 147:3047). The latter have been applied to visualize formation of native CD3ε/δ and CD3ε/γ heterodimers in the cytoplasm of non-lymphoid cells transfected with the corresponding cDNA clones chain (A. Salmeron et al., J. Immunol., 147:3047).

2.2. Lymphocyte Activation by Triggering Surface Receptors

Production of mAbs against lymphocytes has led to the identification of a large number of lymphocyte surface antigens. Expression of these antigens by subsets of lymphocytes has been used to classify T and B cells into specific functional subpopulations and different differentiation stages. More recently, certain of these surface antigens have been recognized as capable of mediating activation signals. Most notably, antibodies directed to CD3 have been used to activate T cells in the absence of antigen (Leo et al., 1987, Proc. Natl. Acad. Sci. U.S.A. 84:1374). In addition, studies of T cell activation have shown that ligand binding to specific coreceptors modifies T cell proliferation and cytokine production initiated by stimulation of the TCR/CD3 complex.

It has been observed that clustering of certain surface antigens as coreceptors results in enhanced T cell activation. Several approaches for using ligands to mediate receptor clustering have been developed. For example, ligands have been immobilized on beads or on plastic surfaces, causing the bound receptors to cluster at the site of contact between the cell and the artificial surface. Receptors have also been clustered together using soluble ligands in the form of bispecific molecules or using a second-step reagent that reacts with two or more monospecific ligands after they have bound to their respective receptors to mediate clustering. Signal transduction experiments and in vitro cell activation experiments using these approaches have generated evidence for functional receptor-coreceptor interactions. However, no acceptable composition for in vivo therapy has been generated.

Aggregation of CD2 with CD3 or CD4 with CD3 has been shown to activate T cells more potently than aggregation of CD3 alone (Ledbetter et al., 1988, Eur. J. Immunol. 18:525-532; Wee et al., 1993, J. Exp. Med. 177:219). Similarly, aggregation of other receptors, including CD18 or CD8 with CD3 enhances signal transduction and activation when compared to aggregation of CD3 alone.

While multiple costimulatory receptors have been identified, knowledge of their relationships to each other, and the spatial and temporal requirements for costimulatory effects on CD3 activation are limited. In one study, co-immobilization of ligands for CD18, CD28, and TCR were studied (Damle et al., 1992, J. Immunol. 149:2541). Indirect immobilization of ICAM1-Ig, B7-Ig and anti-TCR using anti-Ig coated on plastic plates augmented anti-TCR dependent proliferation more than immobilization of ICAM1-Ig or B7-Ig individually. However, ICAM1-Ig was more effective for resting T cells, whereas B7-Ig was more effective for previously activated T cells, implying that the interaction between these coreceptors may be temporal rather than physical.

Although multiple coreceptors modify activation responses through the TCR complex, there is limited information about how these coreceptors work together in aggregate. Clustering of three or more receptors such that each makes a functional contribution to activation signals and overall cellular response has not been well studied.

Studies of B cell activation have also revealed the presence of multiple coreceptors that modify the activation signals and responses initiated by binding to the B cell antigen receptor complex. Notable examples of these receptors include CD19, CD20, CD21, CD22, CD40 and surface immunoglobulin (Ig). Receptor-coreceptor interactions have been demonstrated by using soluble ligands crosslinked together on the cell surface with second step reagents, soluble bispecific molecules such as heteroconjugated antibodies, or combinations of ligands immobilized on a solid surface. Although multiple coreceptors are known, the functional interactions of three or more receptors on B cells have not been reported.

### 3. SUMMARY OF THE INVENTION

The present invention relates to compositions and methods for regulating lymphocyte activation. In particular, the invention relates to compositions and methods for activating T and/or B cells by aggregating three or more cell surface antigens. The activation signals may result in either immune enhancement or immunosuppression.

The invention also relates to inhibition of lymphocyte activation by simultaneous binding to multiple surface receptors and blocking or inhibiting their ability to transmit activation signals and/or by preventing their ability to bind and activate receptors on other cells.

It is an object of the invention to expand the number of T and/or B cells in vitro and in vivo by aggregating three or more surface antigens. Expanded T and B cells are used in adoptive immunotherapy of cancer and infectious diseases such as acquired immunodeficiency syndrome (AIDS). A preferred method for aggregating multiple cell surface antigens in vitro is by adsorption of ligands that bind cell surface antigens and/or antibodies specific for the antigens or their antigen-binding derivatives such as variable domains and complementarity-determining regions (CDRs) of variable domains, onto a solid substrate such as a culture dish or suspendable beads.

While ligands, antibodies or their antigen-binding derivatives may be adsorbed on a biodegradable substrate for in vivo administration, it is preferred that these molecules be combined to form a single soluble multivalent molecule by chemical conjugation or recombinant expression methods. Therefore, it is also an object of the invention to construct a multispecific molecule that simultaneously binds to multiple cell surface antigens. Such multispecific molecule may be immobilized for in vitro lymphocyte activation, or it may be administered as a pharmaceutical composition to a subject for the regulation of lymphocyte activation in vivo. A multispecific molecule may activate lymphocytes by aggregating multiple surface receptors or inhibit lymphocyte activation by interfering with ligand/receptor interactions between T and B cells or between lymphocytes and antigen-presenting cells. A wide variety of uses are encompassed by this aspect of the invention, including but not limited to, treatment of immunodeficiency, infectious diseases and cancer as well as suppression of autoimmunity, hypersensitivity, vascular diseases and transplantation rejection.

The present invention is based, in part, on Applicants' discovery that stimulation of human T cells with immobilized antibodies specific for three T cell surface antigens resulted in enhanced proliferation when compared with stimulation by two immobilized antibodies. Therefore, aggregation of three T cell surface antigens enhanced T cell proliferation. The invention is also based, in part, on Applicants' discovery that llamas immunized with human T cell surface antigens produced antibodies devoid of light chains that bound to such antigens. Since these heavy chain-only antibodies can be generated in llamas against human cell surface antigens, these antibodies and their antigen-binding derivatives are preferred in the construction of multispecific molecules because the lack of light chain participation in antigen binding eliminates the need to include light chains or light chain variable regions. Thus, the use of heavy chain-only antibodies in the construction of multispecific molecules makes the formation of their binding sites less complex. Furthermore, such antibodies contain longer CDRs, especially CDR3, than antibodies composed of heavy and light chains, indicating that CDR peptides derived from heavy chain-only antibodies may be of higher affinity and stability for use in the construction of multispecific molecules.

It is an object of the invention to construct multispecific molecules using heavy chain-only antibodies obtained from the Camelidae family, their variable domains known as V_{HH} or the antigen-binding CDRs derived therefrom. Such multispecific molecules are useful for immunoregulation, based on either stimulation or inhibition of lymphocyte activation. In an effort to enrich for B cells producing this class of V_{HH}-containing antibodies, Applicants also discovered that llama B cells express a human CD40 epitope cross-reactive with an anti-human CD40 antibody, and a subpopulation of CD40⁺ llama cells express heavy chain-only antibodies. Furthermore, the CD40⁺ cells could be activated to proliferate by an anti-CD40 antibody. Hence, it is an object of the invention to enrich for llama B cells that express heavy chain-only antibodies on the basis of their co-expression of CD40 and immunoglobulins without light chains, and to expand their numbers by CD40 stimulation. The expanded cells are particularly useful as a source of mRNA for the construction of libraries of V_{HH} domains and selection of antigen-binding specificities. A novel subclass of such V_{HH} from L. llama are shown in the working examples as lacking a CH1 domain, and their CDR1, CDR2 and CDR3 are not linked by disulfide linkages.

It is also an object of the invention to convert a conventional antibody such as a murine antibody to a heavy chain-only antibody in a process referred to as llamalization. The llamalized antibody retains its original antibody binding specificity without pairing with a light chain.

It is another object of the invention to construct fusion proteins between an antibody variable region or a human antigen and llama constant regions. Such fusion proteins are particularly useful in llama immunization to generate V_{HH} against the non-llama epitopes.

It is yet another object of the invention to generate soluble human CD3 heterodimers.

### 4. BRIEF DESCRIPTION OF THE DRAWINGS

FIG. 1. A schematic description of the isolation of llama V_{HH} polypeptides that bind to cell surface antigens.

FIG. 2. Immobilized mAbs specific for three T cell surface antigens induced enhanced proliferation of human blood T cells.

FIG. 3. Immobilized anti-CD3, anti-CD28 and anti-CD40 mAbs induced enhanced proliferation of T cells.

FIG. 4. Synergy between CD2, CD3 and CD28 activation of purified CD4⁺ T cells as compared to activation of CD8⁺ T cells.

FIGS. 5A & 5B. Stimulation of T cells with immobilized anti-CD2, anti-CD3 and anti-CD28 antibodies resulted in cell growth (5B) in direct correlation with ³H-thymidine incorporation measurements (5A).

FIG. 6. Synergistic effects of mAbs against CD3, CD2 and CD28 co-immobilized on “DYNAL” beads.

FIGS. 7A & 7B. Comparison of co-immobilized and separately immobilized mAbs on T cell proliferation. CD3×CD28=anti-CD3 and anti-CD28 mAbs co-immobilized on same beads. CD3×CD2=anti-CD3 and anti-CD2 mAbs co-immobilized on same beads. CD3+CD28=a mixture of beads coated with anti-CD3 or anti-CD28 mAb. CD3+CD2=a mixture of beads coated with anti-CD3 or anti-CD2 mAb.

FIG. 8. Anti-CD2 in solution or coated on separate beads inhibited co-immobilized anti-CD3 and anti-CD28 in T cell activation.

FIG. 9A-9F. Selective growth of T cells expressing Vβ TCR chains.

FIG. 10A-10F. Llama B cells express CD40 and surface immunoglobulin (Ig), and certain CD40⁺ cells express Ig that do not contain light chain. Llama peripheral blood lymphocytes were unstained (10A), or stained with antibodies: anti-CD40 (10B), anti-CD40 and anti-light chain (1° C.), anti-light chain (10D), anti-CD40 and anti-Ig (10E) and anti-Ig (1° F.).

FIG. 11. Llama B cells proliferated in response to stimulation with an anti-CD40 antibody and CD86 (or B7.2)-expressing transfected CHO cells plus PMA. Results from two different llamas are shown.

FIG. 12. SDS-PAGE analysis of fractionated Llama antibodies. Lane 1 contains IgG1 D (DEAE flowthrough), lane 2 contains IgG1 G (Protein G-bound antibodies eluted at pH 2.7), lane 3 contains IgG2 and IgG3 (Protein G-bound antibodies eluted at pH 3.5) and lane 4 contains IgG3 (Protein G flow through). Lanes 3 and 4 show antibody heavy chain without light chain.

FIG. 13A-13H. Llama heavy chain-only antibodies (IgG2 and IgG3) bound human T cell surface antigens. Jurkat T cells were stained with IgG1 G (13A), IgG1 D (13C), IgG2+IgG3 (13ε) or IgG3 (13G) followed by a second step anti-Ig reagent. Jurkat T cells were also stained with the same antibody fractions (13B, 13D, 13F and 13H), followed by a second step anti-light chain reagent.

FIG. 14. Camelid V_{HH} phage display vector.

FIG. 15. Phage clones, L10 and L11, reacted with a high molecular weight protein expressed on CHO cell surface.

FIG. 16A-16B. Amino acid sequence alignment of Llama V_{HH} polypeptides. 16A shows alignment of several unique hybrid sequences (SEQ ID NOS: 1-9). 16B shows alignment of several complete sequences (SEQ ID NOS: 10-15) which are similar to previously reported camel variable regions.

FIG. 17. Llama constant region sequences (SEQ ID NOS: 16-21).

FIG. 18. Oligonucleotides for antibody 9.3 llamalization (SEQ ID NOS: 22-46). Overlapping oligonucleotides were used to resynthesize 9.3 V_{H} wide type and llamalized version 1(LV1) and version 2 (LV2). The blank spaces for llamalized oligonucleotides are identical to the widetype, thus only altered residues are listed.

FIG. 19. FACS analysis of Jurkat T cells stained by llamalized 9.3 V_{H}.

FIG. 20. Binding activity of various CD3-Ig fusion proteins to anti-CD3 mAbs, G194.

### 5. DETAILED DESCRIPTION OF THE INVENTION

Multiple antigens (or receptors) expressed by lymphocytes work together to regulate cellular activation. In many cases, receptors work together by coming into close proximity or make contact with each other to collectively mediate an activation signal. Under physiological conditions, this process may be controlled by cell-cell contact, where ligands expressed by one cell contact receptors expressed by a second cell, and the receptors are crosslinked and clustered at the site of cell-cell contact. The precise array and order of the receptor contacts may be controlled by the spatial orientation of the ligands and by the inherent ability of the receptors to contact each other at specific sites and in a specific order. The activation signals that are mediated by clustered receptors depend upon intrinsic enzymatic activity of the receptors or of molecules that are directly or indirectly (through linker molecules) associated with each receptor. The clustered receptors allow signaling complexes to form at the cell membrane that result in composite signals dependent upon the precise makeup and orientation of the clustered receptors. Changes in the pattern of receptor clustering result in altered activation states of the resident cell.

The following sections describe compositions and methods for mimicking receptor clustering by aggregating lymphocyte antigens to generate an activation signal. Although the specific procedures and methods described herein are exemplified using immobilized antibodies specific for three T cell antigens, they are merely illustrative for the practice of the invention. Analogous procedures and techniques, as well as functionally equivalent compositions, as will be apparent to those skilled in the art based on the detailed disclosure provided herein are also encompassed by the invention.

5.1. Lymphocyte Surface Antigens

Studies of T and B cell activation have identified a number of cell surface antigens which directly or indirectly mediate activation signals. An “activation signal” as used herein refers to a molecular event which is manifested in a measurable cellular activity such as proliferation, differentiation, cytotoxicity and apoptosis, as well as secretion of cytokines, changes in cytokine profiles, alteration of expression levels or distribution of cell surface receptors, antibodies production and antibody class switching. In addition, an “activation signal” can be assayed by detecting intracellular calcium mobilization and tyrosine phosphorylation of receptors (Ledbetter et al., 1991, Blood 77:1271).

In addition to the TCR/CD3, other molecules expressed by T cells which mediate an activation signal, include but are not limited to, CD2, CD4, CD5, CD6, CD8, CD18, CD25, CD27, CD28, CD40, CD43, CD45, CD45RA, CD45RO, CDw150, CD152 (CTLA-4), CD154, MHC class I, MHC class II, CDw137 (4-1BB), (The Leucocyte Antigen Facts Book, 1993, Barclay et al., Academic Press; Leucocyte Typing, 1984, Bernard et al. (eds.), Springer-Verlag; Leukocyte Typing II, 1986, Reinherz et al. (eds.), Springer-Verlag; Leukocyte Typing III, 1987, McMichael (ed.), Oxford University Press; Leukocyte Typing IV, 1989, Knapp et al. (eds.), Oxford University Press; CD Antigens, 1996, VI Internat. Workshop and Conference on Human Leukocyte Differentiation Antigens. http://www.ncbi.nlm.nih.gov/prow), ICOS (Hutloff et al., 1999, Nature 397:263-266), a cytokine receptor and the like. Cell surface antigens that work together with TCR/CD3 are often referred to as co-receptors in the art.

Specific antibodies have been generated against all of the aforementioned T cell surface antigens, and they are commercially available. Other molecules that bind to the aforementioned T surface antigens include antigen-binding antibody derivatives such as variable domains, peptides, superantigens, and their natural ligands or ligand fusion proteins such as CD58 (LFA-3) for CD2, HIV gp120 for CD4, CD27L for CD27, CD80 or CD86 for CD28 or CD152, ICAM1, ICAM2 and ICAM3 for CD11a/CD18, 4-1BBL for CDw137. Such molecules collectively referred to herein as “binding partners” of surface antigens may be used to deliver or inhibit an activation signal to T cells. For the activation of certain antigens, multiple ligands may be used to achieve the same outcome. For example, B7.1 (CD80), B7.2 (CD86) and B7.3 may be used to activate CD28. B7.3 is a recently identified member of the CD80/CD86 family (GenBank Database Accession No. Y07827). Alignment of the amino acid sequence of B7.3 with those of other family members shows that it is as similar to B7.1 and B7.2 as B7.1 is similar to B7.2.

Activation molecules expressed by B cells, include but are not limited to, surface Ig, CD18, CD19, CD20, CD21, CD22, CD23, CD40, CD45, CD80, CD86 and ICAM1. Similarly, natural ligands of these molecules, antibodies directed to them as well as antibody derivatives may be used to deliver or inhibit an activation signal to B cells.

In a specific embodiment illustrated by examples in Section 6, infra, the present invention demonstrates that aggregation of CD2 and CD3 plus CD28 or CD4 or CD5 enhanced T cell proliferation. In accordance with this aspect of the invention, any three or more up to ten of the aforementioned T and B cell antigens may be bound and aggregated to induce T and B cell activation. For T cell activation, the preferred antigen combinations include CD2 and CD3 with a third antigen being variable, including CD4, CD5, CD6, CD8, CD18, CD27, CD28, CD45RA, CD45RO, CD45, CDw137, CDw150, CD152 or CD154. In addition, it is also preferred that CD2 and CD3 are aggregated with two or three of these surface antigens in any combinations. Examples of these combinations include CD2 and CD3 plus CD4 and CD5 or CD4 and CD28 or CD5 and CD28 or CD8 and CD28 or CDw137 and CD28 or CD4 and CD5 and CD28. For B cell activation, the preferred combinations include CD80 and CD86 with a third antigen being variable, including CD40 or CD56. In addition, CD40 may be aggregated with CD45 and CD86 or with CD19 and CD20. In another preferred embodiment, the antigen combination includes CD3 or TCR and CD28 plus a third antigen described above.

5.2. Methods for Aggregating Multiple Lymphocyte Surface Antigens

One aspect of the present invention relates to methods of aggregating a specific set of three or more antigen combinations to induce lymphocyte activation. A convenient method for aggregating multiple cell surface antigens is by immobilizing “binding partners” of the antigens on a solid substrate such as adsorption on a culture dish, on beads, or on a biodegradable matrix by covalent or non-covalent linkages. In a preferred embodiment, the binding partners are coated on beads, which can be readily separated from cells by size filtration or a magnetic field. While such “binding partners” include natural ligands, binding domains of ligands, and ligand fusion proteins, the preferred embodiments for the practice of this aspect of the invention are antibodies and their antigen-binding derivatives such as Fab, (Fab′)₂, F_{V}, single chain antibodies, heavy chain-only antibodies, V_{HH} and CDRs (Harlow and Lane, 1988, Antibodies, Cold Spring Harbor Press; WO 94/04678). These molecules may be produced by recombinant methods, by chemical synthetic methods or by purification from natural sources. An alternative method to immobilization is cross-linking of three or more antibodies or their antigen-binding derivatives with a secondary antibody that binds a commonly shared epitope. In cases where the molecules are biotinylated, avidin or streptavidin may be used as a second step cross-linking reagent.

In order to adsorb the appropriate antibodies or their antigen-binding derivatives on a solid substrate, the molecules are suspended in a saline such as PBS at a concentration of 1-100 μg/ml. It is preferred that the concentrations are adjusted to 10 μg/ml. After incubation upon a solid surface at 4-37° C. for 1-24 hours, extensive washing is performed to remove the free molecules prior to the addition of cells. Alternatively, antibodies may be covalently conjugated to beads.

Recently, Delamarche et al. (1997, Science 276:779) described the use of microfluidic networks to pattern proteins on a variety of substrates. Such networks may be used to confine an antibody to a specific area of the substrate, so that the cells added thereon are exposed to a different antibody in an orderly fashion as they move through the substrate. As a result, cell surface antigens are aggregated by the antibodies in a sequential order to achieve optimal activation. For example, T cells may be exposed to antibodies to achieve aggregation of surface antigens in the order of CD2→CD3→CD4. Since CD2 and CD4 are located next to CD3, this order of aggregation results in optimal T cell activation. In contrast, aggregation orders of CD2→CD4→CD3 or CD4→CD2→CD3 are expected to be less optimal because in these orders, aggregation of CD2 with CD4 can prevent them from interacting with CD3. The ratios, order and spatial orientation of the binding partners may be adjusted in accordance with a desired outcome.

This aspect of the invention is particularly useful for expansion of lymphocytes in cultures. For the preparation of lymphocytes, peripheral blood mononuclear cells are isolated according to standard procedures and added to the culture dishes containing immobilized antibodies. In addition, T or B cell preparations may be enriched prior to stimulation, using methods well known in the art, including but not limited to, affinity methods such as cell sorting and panning, complement cytotoxicity and plastic adherence. Similarly, distinct T and B cell subsets may be purified using these procedures. Generally, the cells are stimulated for a period of several days to a week followed by a brief resting period and restimulation. Alternatively, the expanded cells may be restimulated every three to fourteen days. In order to facilitate the expansion of cell numbers, growth factors such as IL-2 and IL-4 may be added to the cultures. When the mAbs are attached to a solid surface or beads, stimulatory cytokines may also be similarly attached to the same solid support.

In order to aggregate multiple lymphocyte antigens in vivo, the antibodies and their antigen-binding derivatives may be adsorbed onto a biodegradable substrate made of natural material such as cat gut suture or synthetic material such as polyglycolic acid. However, it is preferred that a single soluble molecule with multiple antigen-binding specificities be used for in vivo administration. In fact, such soluble multispecific molecules are also preferred for in vitro lymphocyte activation when they are immobilized. The following section describes the construction of such molecules.

5.3. Multispecific Molecules that Aggregate Multiple Lymphocyte Surface Antigens

Soluble molecules that bind to multiple cellular target antigens have advantages over molecules immobilized on a particulate matrix for in vivo regulation of the immune system. These advantages include the ability of soluble molecules to rapidly diffuse throughout the immune system, and the formulation of a pharmaceutical composition without an immobilization matrix. Soluble multispecific molecules have advantages over combinations of monospecific molecules in specificity and avidity, resulting in increased potency and effectiveness. A multispecific molecule also possesses an increased target cell specificity even though individual components lack specificity for a particular cell type. Several low affinity (<50 nm) binding sites specific for distinct target antigens may be fused in tandem to form a multispecific protein with increased binding avidity for the cells expressing all target antigens. For example, even though CD18 is expressed by all lymphocytes, a multispecific molecule composed of a CD18-binding partner may still exhibit lymphocyte subset specificity because a lymphocyte subset expressing CD18 and not the other target antigens of the multispecific molecule would not bind the molecule with high avidity.

Regulation of the immune system includes lymphocyte activation, incomplete stimulation signals that do not result in full activation, causing apoptosis or anergy of lymphocytes, and blockade of multiple receptor-ligand interactions simultaneously. In addition, activation of cells to secrete inhibitory cytokines could result in active suppression of specific responses. In that regard, T cells may be activated to become “TH₂”-like cells and induced to secrete TGFβ and IL-10 which suppress immune responses by IL-4 production plus a signal to TCR/CD3. Cytokines such as IL-4 may be covalently attached to a solid support or otherwise immobilized with antibodies or ligands to induce TH₂ T cell differentiation. A multispecific molecule may be constructed between a low affinity (<100 nm) CD3 binding site and binding sites for CD2 and CD4 for that purpose. For T cell activation, a preferred multispecific molecule is composed of binding partners that aggregate CD2, CD3 and CD28. Other T cell activation multispecific molecules are composed of binding partners that aggregate CD2 and CD3 or CD3 and CD28 with a third variable antigen such as those described in Section 5.1., supra.

Also within the scope of the present invention are soluble multispecific molecules that inhibit T and B cell activation. Such inhibitory molecules can bind two, three and up to ten antigens on the same surface simultaneously and inhibit the delivery of an activation signal through these antigens. An example of one such multispecific molecule binds to CD80, CD86, and CD40 on antigen presenting cells and B cells, and interferes with activation of the CD28 pathway and the CD40 pathway simultaneously. A bispecific inhibitor of the CD28 and CD40 pathways binds to CD28 and CD154 (the CD40 ligand) on T cells, blocking activation of CD28 and preventing CD154 from activating CD40. Other T cell inhibitory bispecific molecules target CD20 and CD40 or CD2 and CD4 or CD28 and CD45 or CD2 and CD154. Trispecific inhibitory molecules target CD2 and CD28 and CD45 or CD2 and CD4 and CD45 or CD2 and CD4 and CD28 or CD2 and CD27 and CD28.

Soluble multispecific molecules that bind to multiple B cell receptors and enhance activation signals are particularly advantageous for induction of apoptosis of malignant B cells. Such multispecific molecules also have advantages in specific targeting since they are expected to bind more strongly to a cell that expresses all of the receptors and bind less well to any cell that expresses only one or a subset of the receptors recognized by the multispecific molecules. A preferred multispecific molecule binds to CD19, CD20, and CD40 receptors simultaneously, and generates activating signals through these receptors to result in apoptosis of malignant B cells. Bispecific and multispecific B cell inhibitory molecules may target CD80 and CD40 or CD86 and CD40 or CD80 and CD86 or CD80 and CD86 and B7-3 on B cells or antigen presenting cells.

A multispecific molecule may be produced by chemical conjugation of multiple binding partners that bind cell surface antigens or by recombinant expression of polynucleotides that encode these polypeptides. In an effort to reduce the complexity of ligating multiple polypeptide chains such as those seen in antibodies or their coding sequences, it is preferred that single chain polypeptides of low molecule weight be used as binding partners to construct multispecific molecules. In that connection, it has been reported in WO94/04678 that camels secrete antibodies devoid of light chains. The variable domain of such heavy chain-only antibodies referred to as V_{HH} are fused directly to a hinge region which is linked to the CH2 and CH3 domains. The absence of a CH1 domain in the heavy chains prevents formation of disulfide linkages with light chains.

Heavy chain-only antibodies are particularly suitable for use in the construction of multispecific molecules because there is no participation in antigen binding by light chains. V_{HH} domains of these antibodies are even more suitable because the removal of their constant domains reduces non-specific binding to Fc receptors. Section 8, infra, demonstrates that V_{HH} domains of L. llama contain CDR3 that are longer than CDRs in conventional antibodies, and the CDRs of a particular subclass (hybrid subclass) of these V_{HH} sequences do not form disulfide linkages with other CDRs in the same variable domain. Therefore, these CDRs may be more stable and independent in antigen binding, and can be readily expressed to result in proper folding. The unique features of this class of CDRs render them particularly suitable for use in the construction of multispecific molecules. The CDRs in these antibodies can be determined by methods well known in the art (U.S. Pat. No. 5,637,677), and used for the production of multispecific molecules.

Variable region sequences from L. llama are similar to sequences in the human VH₃ family of variable domains (Schroeder et al., 1989, Int. Immunol. 2:41-50). In order to reduce immunogenicity of V_{HH} molecules for use in a human recipient, amino acids in non-CDR or exposed framework sites may be altered on the basis of their differences from human VH₃ residues. Crystal structure of a camel V_{HH} can be used as a guide to prioritize residue changes based on the extent of exposure (Desmyter et al., 1996, Nat. Struct. Biol. 3:803-811). Other methods of predicting immunogenicity of residues may also be used (i.e. hydrophilicity or MHC binding motifs) to guide the choice of residue substitutions. Residues within or adjacent to CDRs that are critical for antigen binding should be preserved in order to avoid a reduction in binding avidity. Similarly, framework residues that are identified as important in eliminating the hydrophobic V_{L}-V_{H} interface should be preserved for optimal folding and expression of V_{HH} molecules.

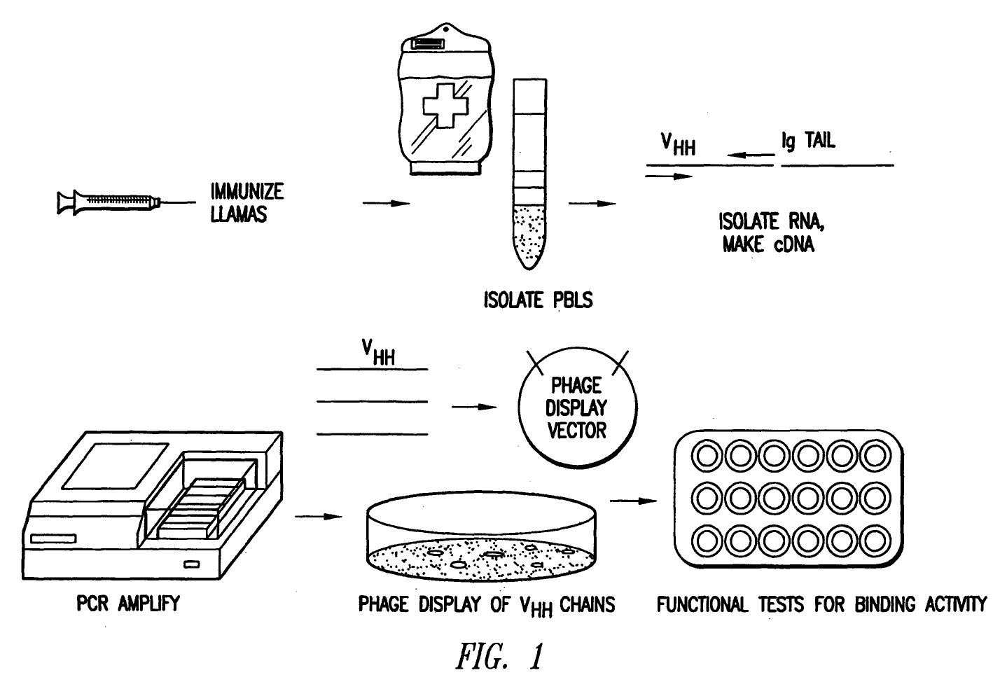

In a specific embodiment illustrated by examples in Section 7, infra, heavy chain-only antibodies purified from a llama immunized with human T cells bound to T cell surface antigens. FIG. 1 provides a scheme for rapidly screening and selecting V_{HH} domains with cell surface antigen-binding specificities. For the generation of V_{HH} domains, animals belonging to the Camelidae family are used as hosts for immunization with a purified antigen, fusion protein between a human cell surface antigen and llama antibody constant region, or cells expressing an antigen of interest. These hosts, include but are not limited to, old world camelids such as Camelus bactrianus and C. dromaderius, and new world camelids such as Llama paccos, L. glama, L. vicugna and L. llama. After immunization, peripheral blood leukocytes or mononuclear cells from other lymphoid tissues such as lymph nodes and spleens are isolated by density gradient centrifugation and their cDNA obtained by reverse transcription/polymerase chain reaction as described in Section 8.1.2., infra. Phage display technology may be used to express the isolated V_{HH} fragments for the selection of antigen-specific binding V_{HH} (U.S. Pat. Nos. 5,223,409; 5,403,484 and 5,571,698). Examples of a number of isolated V_{HH} sequences from L. llama are shown in Section 8 infra.

Heavy chain-only antibodies may also be produced by conventional hybridoma technology originally described by Koehler and Milstein, 1975, Nature 256:495-497. Monoclonal heavy chain-only antibodies may be proteolytically cleaved to produce V_{HH} domains.

Isolated V_{HH} domains or multispecific molecules composed of V_{HH} domains may be fused with a second molecule with biologic effector functions. For example, they may be fused with a toxin such as pseudomonas exotoxin 40 (PE40) for specific delivery to kill unwanted cells such as cancer cells or autoreactive T cells. They may also be fused with cytokines to deliver signals to specific cell types, or with extracellular domains of receptors or receptor binding domains to combine receptor specificity with the specificity of V_{HH}. In addition, they may be fused with Ig Fc domains, Ig Fc domains containing specific mutations (U.S. Pat. No. 5,624,821), or portions of Fc domains to construct chimeric antibody derivatives. They may be fused with intracellular targeting signals to allow specific binding to antigens located inside cells. They may be fused with proteins that act as enzymes or that catalyze enzyme reactions. In addition, the multispecific molecules may be expressed as genes to improve and/or simplify gene therapy vectors.

5.3.1. Construction of Multispecific Molecules

A preferred method of making soluble multispecific molecules is the fusion of multiple camelid V_{HH} variable regions, each specific for a chosen cellular target antigen. Llamas are a preferred camelid species as a source of such variable regions because they are readily available. The functional activity of a multispecific molecule depends upon the composition, spacing, and ordering of the binding sites of the variable regions. Composition of the binding sites would depend upon the specificity of the individual V_{HH} used and the number of each V_{HH} in the molecule. V_{HH} target specificity may include one or more V_{HH} binding domains against a single receptor fused to other V_{HH} domains targeted to a second or a third receptor. Molecules that target two or more epitopes on only one receptor are within the scope of the invention. These molecules have increased binding avidity for the target and crosslink a single receptor on the cell surface by binding to multiple epitopes. The order of V_{HH} domains and receptor epitopes may be important for driving intra- or inter-receptor binding patterns. The spacing of the binding sites would depend upon the choices of linkers used between V_{HH} domains. Linker length and flexibility are both factors that would control spacing between binding domains. Ordering of the binding sites would be controlled by ordering the V_{HH} domains within the fusion protein construct.

Camelid V_{HH} domains with binding specificity for lymphocyte antigens or CDRs derived from them could be linked together in tandem arrays, either genetically or chemically. If the arrays are genetically linked, fusion proteins are created with multiple antigen binding specificities in a single molecule. In the preferred multispecific structure, the linked molecules should result in the same spectrum of activity, so that blocking, inhibitory molecules are linked to create a more potent immunosuppressive agent. Similarly, agonists that aggregate and stimulate the bound receptors would be linked in order to achieve more potent activation of the lymphocytes bound through their receptors for potential ex vivo cell therapy applications with soluble or immobilized molecules.

The linkers used in either the suppressive or activator molecules might take one of several forms, with the preferred linkers containing repeated arrays of the amino acids glycine and serine. As an example, (gly₄ser)₃ or (gly₃ser₂)₃ are two preferred choices of linker between antigen binding domains. This linker might need to be lengthened in order to achieve optimal binding of the flanking V_{HH} domains, depending on the size and spacing of the target antigens on the cell surface.

The configuration of V_{HH} domains might be altered in successive embodiments to determine which structures give the optimal biological effect. In a trispecific molecule, the V_{HH} domain in the center of the molecule might be most constrained and therefore might have an apparent decrease in avidity for its target relative to the two flanking domains. Similarly, some V_{HH} domains might be more sensitive to amino versus carboxy terminal fusions. The suppressive effects of a CD80-CD86-CD40 structure might therefore differ from a CD80-CD40-CD86, CD40-CD80-CD86, CD40-CD86-CD80, or a CD86-CD40-CD80 type molecule.

5.3.2. Production of Multispecific Molecules by Chemical Conjugation Methods

A multispecific molecule may be constructed by chemical conjugation of three or more individual molecules. Glennie & Trutt (1990, Bispecific Antibodies and Targeted Cellular Cytotoxicity, pp. 185, Romet-Lemonne (eds.)) describe a method for constructing trispecific antibodies using chemical methods. Briefly, trispecific F(ab′)₃ can be constructed by first preparing a bispecific F(ab′)₂ derivative containing the two Fab′ arms, and linking it to a third Fab′ arm. F(ab′)₂ from two antibodies are first reduced to yield Fab′(SH) and all the available sulfhydryl groups on one antibody Fab′(SH) are maleimidated with a bifunctional cross-linker o-phenylenedimaleimide (o-PDM) followed by reacting Fab′ (mal) with the Fab′ (SH) under conditions which favor a reaction between SH and maleimide groups while minimizing the reoxidation of SH-groups. After isolating the bispecific F(ab)₂ by column chromatography, it is reduced and linked to Fab′(mal) from a third antibody. All derivatives are reduced and alkylated to safeguard against any minor untoward products which may form by disulfide exchange or oxidation of SH-groups during an overnight incubation. All multispecific Fab′ derivatives are passed through a highly specific anti-mouse Fcy immunosorbent to remove any trace amounts of parent monoclonal IgG which may have escaped with the parent F(ab′)₂ fragments following fractionation of the digest mixture.

The aforementioned protocol was originally designed for linking Fab fragments from mouse IgG to form trispecific (Fab′)₃ through tandem thioether linkages of the hinge-region sulfhydryl groups using the cross-linker o-PDM. However, this method may be adjusted for linking any three or more molecules for the construction of multispecific molecules, including, but not limited to, ligands, binding domains of ligands, antibodies, Fv, V_{HH} and CDR.

5.3.3. Production of Multispecific Molecules by Recombinant Methods

The multispecific molecules containing V_{HH} domains will show improvements in expression levels in many cell systems, including bacterial expression, yeast expression, insect expression and mammalian expression systems. The characteristic changes in V_{HH} domains allow expression without requiring pairing with a light chain variable region through a strong hydrophobic interaction. Conventional variable regions are not secreted or expressed on the cell surface without pairing with a second variable region to mask the hydrophobic variable region interface. Therefore the expression of variable regions is linked to the hydrophobic interface that mandates pairing with a second variable region. V_{HH} domains are expressed individually and should be expressed at much higher levels because of the alterations in hydrophobic residues that restrict expression.

The multispecific molecules containing V_{HH} domains also will express better because they can be folded into their active conformations more easily. This will be a significant advantage in bacterial expression where active molecules may be expressed without requiring refolding procedures in vitro after expression of denatured protein. Improved folding may also help improve expression in mammalian cells.

Improvements in expression levels will meet an important need for production of antibody-based therapeutics. High costs of goods have been a significant limitation for commercialization of products based on antibody binding sites where molecules may be active in vivo but require high levels of protein for therapeutic efficacy (sometimes exceeding 1 gram per patient). In fact, it is likely that high costs associated with expression currently represent the greatest barrier to success with antibody based products.

For recombinant production, a contiguous polynucleotide sequence containing coding sequences of multiple binding partners is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the encoded product. Depending on the expression system used, the expressed product is then isolated by procedures well-established in the art. Methods for recombinant protein and peptide production are well known in the art (see, e.g., Maniatis et al., 1989, Molecular Cloning A Laboratory Manual, Cold Spring Harbor Laboratory, N.Y.; and Ausubel et al., 1989, Current Protocols in Molecular Biology, Greene Publishing Associates and Wiley Interscience, N.Y.).

The published crystal structure (Desmyter et al., 1996, Nat. Struct. Biol. 3:803-811) of a camelid V_{HH} molecule indicates that the amino and carboxy termini of the V_{HH} molecule are exposed to solvent on different sides of the molecule, the desired configuration for constructing multispecific fusion proteins. Multispecific V_{HH} molecules are constructed by linking the cDNAs encoding one V_{HH} to a second V_{HH} through a spacer cDNA encoding an amino acid linker molecule. Adding another V_{HH} and linker to this bispecific, and continuing this process to gradually build an array of binding sites, results in a multispecific molecule. By including the appropriate unique restriction sites at each end of the V_{HH} and linker cassettes, the molecules can be assembled in any plasmid vector with the appropriate restriction site polylinker for such sequential insertions. Alternatively, a new polylinker may be constructed in an existing plasmid that encodes several restriction sites interspersed with DNA encoding the amino acid linkers for at least two of the junctions between V_{HH} molecules. Some of the linkers include (gly₄ser)₃, (gly₃ser₂)₃, other types of combinations of glycine and serine (glyₓser_{y})_{z}, hinge like linkers similar to those attached to the llama V_{HH} domains (including some or all portion of the region between amino acids 146-170) which include sequences encoding varying lengths of alternating PQ motifs (usually 4-6) as part of the linker, linkers with more charged residues to improve hydrophilicity of the multispecific molecule, or linkers encoding small epitopes such as molecular tags for detection, identification, and purification of the molecules.

A preferred embodiment of the present invention includes PCR amplification of V_{HH} molecules targeted to CD80, CD86, and CD40, each with unique, rare restriction sites at the ends of the cDNAs. An expression plasmid is created with a polylinker into which complementary oligonucleotides encoding two or more of the amino acid linkers outlined above have been inserted and annealed. At each end of the inserted oligonucleotides, the restriction site matches that found on the amino or carboxy terminus (5′ or 3′ end) of one of the V_{HH} cassettes. Multispecific molecules can then be assembled by successive digestion and ligation of the oligonucleotide-polylinker plasmid with the individual V_{HH} cassettes.

A variety of host-expression vector systems may be utilized to express a multispecific molecule. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter; cytomegalovirus (CMV) promoter) may be used; when generating cell lines that contain multiple copies of expression product, SV40-, BPV- and EBV-based vectors may be used with an appropriate selectable marker.

In cases where plant expression vectors are used, the expression of sequences encoding a multispecific molecule may be driven by any of a number of promoters. For example, viral promoters such as the 35S RNA and 19S RNA promoters of CaMV (Brisson et al., 1984, Nature 310:511-514), or the coat protein promoter of TMV (Takamatsu et al., 1987, EMBO J. 6:307-311) may be used; alternatively, plant promoters such as the small subunit of RUBISCO (Coruzzi et al., 1984, EMBO J. 3:1671-1680; Broglie et al., 1984, Science 224:838-843) or heat shock promoters, e.g., soybean hspl7.5-E or hspl7.3-B (Gurley et al., 1986, Mol. Cell. Biol. 6:559-565) may be used. These constructs can be introduced into plant cells using Ti plasmids, R1 plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In one insect expression system that may be used to produce the molecules of the invention, Autographa californica nuclear polyhidrosis virus (AcNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (e.g., see Smith et al., 1983, J. Virol. 46:584; Smith, U.S. Pat. No. 4,215,051). Further examples of this expression system may be found in Current Protocols in Molecular Biology, Vol. 2, Ausubel et al., eds., Greene Publish. Assoc. & Wiley Interscience.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. (e.g., See Logan & Shenk, 1984, Proc. Natl. Acad. Sci. (USA) 81:3655-3659). Alternatively, the vaccinia 7.5 K promoter may be used, (see, e.g., Mackett et al., 1982, Proc. Natl. Acad. Sci. (USA) 79:7415-7419; Mackett et al., 1984, J. Virol. 49:857-864; Panicali et al., 1982, Proc. Natl. Acad. Sci. 79:4927-4931).

A multispecific molecule can be purified by art-known techniques such as high performance liquid chromatography, ion exchange chromatography, gel electrophoresis, affinity chromatography and the like. The actual conditions used to purify a particular molecule will depend, in part, on factors such as net charge, hydrophobicity, hydrophilicity, etc., and will be apparent to those having skill in the art.

For affinity chromatography purification, any antibody which specifically binds the molecule may be used. For the production of antibodies, various host animals, including but not limited to rabbits, mice, rats, etc., may be immunized by injection with a multispecific molecule or a portion thereof. The molecule or a peptide thereof may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

5.4. Uses of Activated Lymphocytes Following Multiple Surface Antigen Aggregation

Lymphocytes are activated in culture by aggregation of multiple surface antigens in accordance with the method of the invention. The activated cells may be used in adoptive therapy of infectious diseases, particularly viral infections such as AIDS, and cancer. Activated cells may secrete cytokines or have other effector mechanisms that suppress responses to autoantigens or transplants, and may therefore be useful for treatment of autoimmune diseases and transplant rejection. In addition, multi specific molecules that aggregate multiple antigens may be administered directly into a subject to augment immune responses against an infectious agent such as a virus or against tumor cells. Furthermore, such molecules may deliver an apoptotic signal to T and B cell tumors to directly induce tumor destruction. Alternatively, multispecific molecules may be used as inhibitors of immune responses by interfering with antigen presentation or T cell/B cell interactions. These molecules are useful for treatment of autoimmunity, and hypersensitivity as well as prevention of transplantation rejections.

5.4.1. Formulation and Route of Administration

A multispecific molecule of the invention may be administered to a subject per se or in the form of a pharmaceutical composition. Pharmaceutical compositions comprising a multispecific molecule of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the active ingredient into preparations which can be used pharmaceutically. Proper formulation is dependent upon the route of administration chosen.

For topical administration, a multispecific molecule of the invention may be formulated as solutions, gels, ointments, creams, suspensions, etc. as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration such as aerosol, inhaler and nebulizer.

For injection, a multispecific molecule of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

Alternatively, a multi specific molecule may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, a multispecific molecule can be readily formulated by combining with pharmaceutically acceptable carriers well known in the art. Such carriers enable a multispecific molecule of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, a multispecific molecule may take the form of tablets, lozenges, etc. formulated in conventional manner.

For administration by inhalation, a multispecific molecule for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

A multispecific molecule may also be formulated in rectal or vaginal compositions such as suppositories or retention enemas, e.g, containing conventional suppository bases such as cocoa butter or other glycerides.

In addition to the formulations described previously, a multispecific molecule may also be formulated as a depot preparation. Such long acting formulations may be administered by implantation (for example subcutaneously or intramuscularly) or by intramuscular injection. Thus, for example, a multispecific molecule may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver a multispecific molecule of the invention. Certain organic solvents such as dimethylsulfoxide also may be employed, although usually at the cost of greater toxicity. Additionally, a multispecific molecule may be delivered using a sustained-release system, such as semipermeable matrices of solid polymers containing the therapeutic agent. Various sustained-release materials have been established and are well known by those skilled in the art. Sustained-release capsules may, depending on their chemical nature, release a multispecific molecule for a few weeks up to over 100 days. Depending on the chemical nature and the biological stability of the therapeutic reagent, additional strategies for protein stabilization may be employed.

As a multispecific molecule of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

5.4.2. Effective Dosages

A multispecific molecule of the invention will generally be used in an amount effective to achieve the intended purpose. For use to activate or suppress an immune response mediated T cells and/or B cells, a multispecific molecule of the invention, or pharmaceutical compositions thereof, are administered or applied in a therapeutically effective amount. By therapeutically effective amount is meant an amount effective to ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of a multispecific molecule which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day.

In cases of local administration or selective uptake, the effective local concentration of a multispecific molecule may not be related to plasma concentration. One having skill in the art will be able to optimize therapeutically effective local dosages without undue experimentation.

The amount of a molecule administered will, of course, be dependent on the subject being treated, on the subject's weight, the severity of the affliction, the manner of administration and the judgment of the prescribing physician.

The therapy may be repeated intermittently while symptoms are detectable or even when they are not detectable. The therapy may be provided alone or in combination with other drugs.

5.4.3. Toxicity

Preferably, a therapeutically effective dose of a multispecific molecule described herein will provide therapeutic benefit without causing substantial toxicity.

Toxicity of a multispecific molecule described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Molecules which exhibit high therapeutic indices are preferred. The data obtained from these cell culture assays and animal studies can be used in formulating a dosage range that is not toxic for use in human. The dosage of a multispecific molecule described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch.1, p.1).

5.5. Transgenic Animals that Express Llama V_{Hh}

The V_{HH} gene sequences isolated by the methods disclosed herein can be expressed in animals by transgenic technology to create founder animals that express llama V_{HH} (U.S. Pat. No. 5,545,806; WO98/24893). Animals of any species, including, but not limited to, mice, rats, rabbits, guinea pigs, pigs, micro-pigs, goats, sheep, and non-human primates, e.g., baboons, monkeys, and chimpanzees may be used to generate llama V_{HH}-expressing transgenic animals. The term “transgenic,” as used herein, refers to animals expressing coding sequences from a different species (e.g., mice expressing llama gene sequences).

Any technique known in the art may be used to introduce V_{HH} transgenes into animals to produce the founder lines of transgenic animals. Such techniques include, but are not limited to, pronuclear microinjection (Hoppe and Wagner, 1989, U.S. Pat. No. 4,873,191); retrovirus-mediated gene transfer into germ lines (Van der Putten, et al., 1985, Proc. Natl. Acad. Sci., USA 82:6148-6152); gene targeting in embryonic stem cells (Thompson, et al., 1989, Cell 56:313-321); electroporation of embryos (Lo, 1983, Mol. Cell. Biol. 3:1803-1814); and sperm-mediated gene transfer (Lavitrano et al., 1989, Cell 57:717-723) (see Gordon, 1989, Transgenic Animals, Intl. Rev. Cytol. 115, 171-229). Any technique known in the art may be used to produce transgenic animal clones containing V_{HH} transgenes, for example, nuclear transfer into enucleated oocytes of nuclei from cultured embryonic, fetal or adult cells induced to quiescence (Campbell, et al., 1996, Nature 380:64-66; Wilmut, et al., Nature 385:810-813).

The present invention provides for transgenic animals that carry the V_{HH} transgenes in all their cells, as well as animals that carry the transgenes in some, but not all their cells, i.e., mosaic animals. The V_{HH} may be integrated as individual gene segments or in concatamers, e.g., head-to-head tandems or head-to-tail tandems. The V_{HH} transgenes may also be selectively introduced into a particular cell type such as lymphocytes by following, for example, the teaching of Lasko et al. (1992, Proc. Natl. Acad. Sci. USA 89:6232-6236). The regulatory sequences required for such a cell-type specific activation will depend upon the particular cell type of interest, and will be apparent to those of skill in the art. When it is desired that the transgenes be integrated into the chromosomal site of the endogenous variable region genes, gene targeting is preferred. Briefly, when such a technique is to be utilized, vectors containing some nucleotide sequences homologous to the endogenous genes are designed for the purpose of integrating, via homologous recombination with chromosomal sequences, into and disrupting the function of the nucleotide sequences of the endogenous genes. The transgenes may also be selectively introduced into a particular cell type, thus inactivating the endogenous genes in only that cell type, by following, for example, the teaching of Gu, et al. (1994, Science 265: 103-106). The regulatory sequences required for such a cell-type specific inactivation will depend upon the particular cell type of interest, and will be apparent to those of skill in the art.

Once transgenic animals have been generated, the expression of the llama V_{HH} may be assayed utilizing standard techniques. Initial screening may be accomplished by Southern blot analysis or PCR techniques to analyze animal tissues to assay whether integration of the V_{HH} has taken place. The level of mRNA expression of the V_{HH} in the tissues of the transgenic animals following immunization of an antigen may also be assessed using techniques that include, but are not limited to, Northern blot analysis of tissue samples obtained from the animal, in situ hybridization analysis, and RT-PCR. Samples of V_{HH}-expressing tissue, may also be evaluated immunocytochemically using antibodies specific for llama variable region epitopes.

Various procedures known in the art may be used for the production of V_{HH} to any antigen by immunizing transgenic animals with an antigen. Mice are preferred because of ease of handling and the availability of reagents. Such antibodies include, but are not limited, to polyclonal, monoclonal, chimeric, humanized, single chain, anti-idiotypic, antigen-binding antibody fragments and fragments produced by a variable region expression library.

Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

MAbs may be prepared by using any technique which provides for the production of antibody molecules by continuous cell lines in culture. These include but are not limited to the hybridoma technique originally described by Kohler and Milstein, (Nature, 1975, 256:495-497). Such antibodies may be heavy chain-only antibodies and of any immunoglobulin class including, but not limited to, IgG, IgM, IgE, IgA, IgD and any subclass thereof.

The invention having been described, the following examples are offered by way of illustration and not limitation.

6. Example: Immobilized Antibodies Specific for Three T Cell Surface Antigens Enhanced Human T Cell Proliferation

6.1. Materials and Methods

6.1.1. Stimulation of Human T Cell Proliferation

Mononuclear cells were isolated from human peripheral blood by centrifugation on “FICOLL”. Monocytes were depleted by two rounds of adherence to plastic. The mononuclear cells were then stimulated in 96-well Costar flat-bottom microtiter plates at 50,000 cells per well containing immobilized antibodies. The antibodies were immobilized by incubating purified antibody mixtures in phosphate buffered saline (PBS) in the wells at 100 μl/well for 3 hr at 37° C., followed by washing away of the unbound antibodies from the wells prior to addition of cells. Antibody concentrations were 10 μg/ml of anti-CD3, 10 μg/ml of anti-CD2, and varying concentrations of a third antibody as indicated. Proliferation was measured in quadruplicate wells by incorporation of ³H-thymidine during the last 18 hours of a 4 day culture. Means are shown, and standard errors are less than 15% of the mean at each point.

6.1.2. Anti-T Cell Antibodies

MAb anti-CD3, OKT3, was obtained from ATCC (ATCC CRL-8001). MAb anti-CD28, B-T3, was purchased from Diaclone (Besancon, France). MAb anti-CD2, 9.6, and anti-CD28 antibody, 9.3, were provided by John Hansen (FHCRC, Seattle, Wash.). Anti-CD4, OKT4, was obtained from the ATCC (ATCC CRL-8002). MAb anti-CD5, 10.2, was provided by John Hansen (FHCRC, Seattle, Wash.). Control mAb was L6. Anti-CD40 mAb is described by Clark and Ledbetter (1986, Proc. Natl. Acad. Sci. U.S.A. 83:4494-4498). Anti-CD18 mAb is described by Beatly et al. (1983, J. Immunol. 131:2913-2918).

6.1.3. T Cell Subset Separation

T cells were isolated from peripheral blood by centrifugation on “FICOLL”, followed by separation into CD4⁺ or CD8⁺ subsets by depletion of monocytes, B cells, NK cells, and either CD4 or CD8 cells. Cell depletion was performed using mAbs to CD14, CD20, CD11b, and CD8 or CD4 followed by removal of antibody-bound cells using magnetic beads coated with anti-mouse IgG. CD4⁺ or CD8⁺ T cells were >95% pure after the depletion step when analyzed by flow cytometry. Cells were cultured in antibody-coated microtiter plates at 5×10⁴ for 4 days, and proliferation was measured by incorporation of ³H-thymidine for the final 12 hours of culture. Microtiter plates contained immobilized antibodies as indicated, including the control, nonbinding L20 antibody in some wells to equalize the total protein concentration for immobilization. Antibodies were immobilized by incubation at 10 μg/ml each for 18 hr at 37° C., followed by removal of unbound protein by extensive washing.

6.1.4. Anti-TCR Variable Region Antibodies

MAbs specific for TCR Vβ8 (Pharmingen 3313 1A), Vβ9 (Pharmingen 3313 1B), Vβ14 (Coulter Im. 1557), and Vβ20 (Coulter Im. 1561) were immobilized on culture plates using a two-step procedure. Purified goat anti-mouse (Capel) antibody was immobilized first, followed by washing and blocking before addition of the anti-Vβ mAb plus anti-CD28. Cell growth was observed, and after 9 days, the proliferating cells were transferred to new culture plates containing 5 U/mL interleukin-2 (R&D, Inc., Minneapolis, Minn.). Five days later, on day 14, the cells were analyzed by flow cytometry for expression of TCR Vβ specificity using a secondary fluorescein-conjugated anti-mouse IgG reagent (Biosource).

6.1.5. Antibody Coupling to Beads for Cell Stimulation

A suspension of 2.8 ml “DYNAL” beads (Oslo, Norway), M-450 tosyl activated, at 4×10⁸ beads/ml were washed three times, each with four me of 0.1M sodium borate, pH9.5, using a magnet for buffer removal. The beads were then suspended in 1.5 ml of borate buffer. To 200 μl (1.8×10⁸ beads) of bead suspension was added a mixture of 140 μl borate buffer, 30 μg of a given antibody to be coupled, and PBS. The volume of added PBS was adjusted such that the final volume of the reaction mixture was 400 μl. All possible combinations of antibodies to CD3 (OKT-3), CD28 (9.3), and CD2 (9.6) were coupled. The antibodies were allowed to react with the beads for approximately 20 hr at 37° C. on a rotator. This was followed by removal of unreacted antibody with a magnet. The bead preparations were then washed three times with 1 ml PBS containing 0.1% (wt:vol) sodium azide and three times with PBS containing 3% (vol:vol) human serum, 5 mM EDTA, and 0.1% (wt:vol) sodium azide (storage buffer). The last of the three washes in storage buffer was done for 30 minutes at ambient temperature on a rotator. All the bead preparations were then incubated with storage buffer for approximately 31 hr at 4° C. on a rotator. This was followed by re-suspension of each of the preparations in 1.0 ml storage buffer.

Peripheral blood lymphocytes were isolated by density centrifugation. The lymphocytes were adhered to plastic in RPMI with 2% FCS. Cells were pelleted and plated in 96-well flat-bottom plates at a density of 2.5×10⁵/ml. Dynal beads conjugated with mAbs were then plated with the cells at a ratio of 3 beads: 1 cell. Cells were incubated at 37° C. and 5% CO₂ for 5 days. One μCi/well of ³H-thymidine was then added to the wells and incubated overnight. Cultures were harvested on a glass filter mat and cpm measured.

6.2. Results

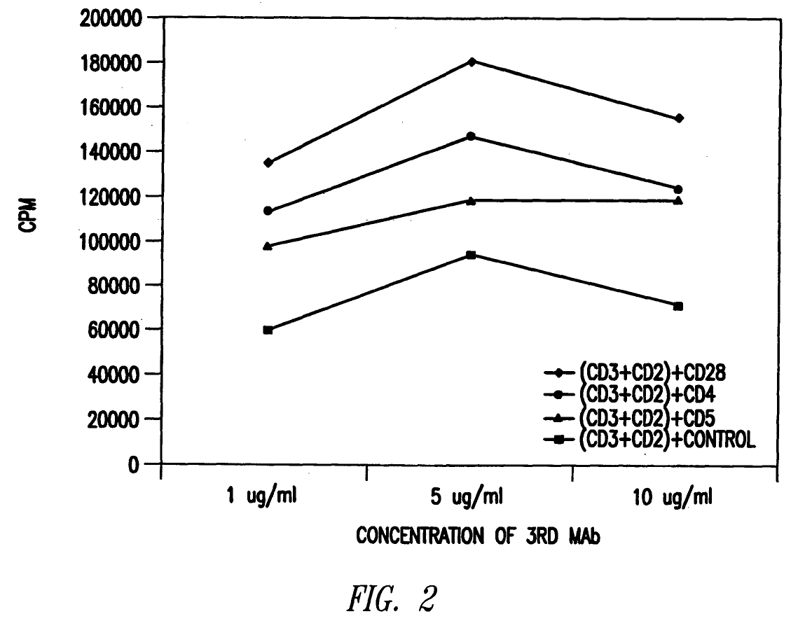

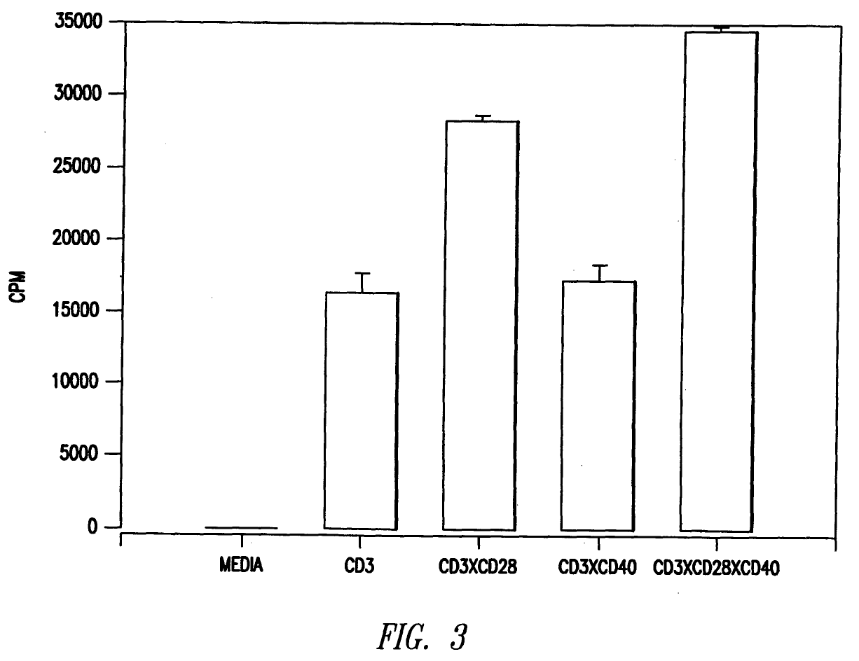

Human T cells were isolated from peripheral blood of normal donors and stimulated in vitro with immobilized mAbs directed to three T cell surface antigens. Antibodies specific for CD2 and CD3 plus a third antibody, such as anti-CD28, anti-CD4 or anti-CD5, were co-immobilized by adsorption on the surface of culture plates, followed by incubation with T cells in culture media. T cell proliferation was assayed as a measure of T cell activation. The combination of three immobilized antibodies enhanced T cell proliferation when compared with the combined use of immobilized anti-CD2, anti-CD3 antibodies and a third control antibody, L6, specific for an antigen not expressed by T cells (FIG. 2). In particular, the combination of anti-CD2, anti-CD3 and anti-CD28 produced the highest level of T cell proliferation at all concentrations tested. Three immobilized antibodies induced greater cellular proliferation than the same antibodies presented in solution or two immobilized antibodies plus a third antibody in solution. Co-immobilized anti-CD3 and anti-CD28 plus anti-CD18 mAbs also induced greater T cell proliferation than the combination of two of the three antibodies. Additionally, co-immobilized anti-CD3, anti-CD28 and anti-CD40 mAbs enhanced proliferation of purified T cells (FIG. 3). It is noted that CD40 is expressed by activated T cells as well as antigen presenting cells. Therefore, aggregation of three T cell surface antigens by co-immobilized antibodies enhanced T cell activation. Immobilized antibodies may be used to expand T cell and B cell numbers in culture as well as inducing cellular differentiation. The activated cells can be separated from the immobilized antibodies more easily than from antibodies added in solution so that injection of antibodies bound to cells into a recipient can be minimized when the cells are harvested for use in adoptive therapy.

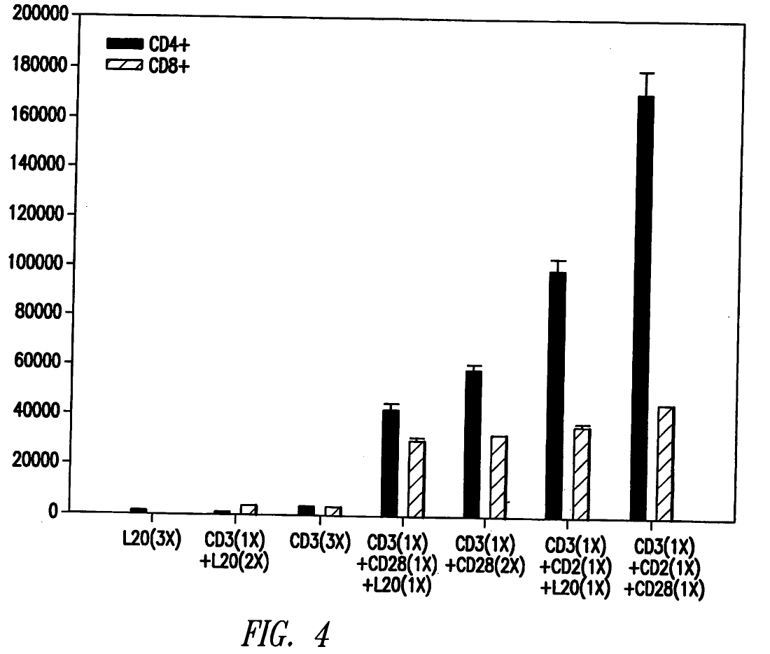

When purified CD4⁺ or CD8⁺ T cells were incubated with immobilized anti-CD3 antibody, cellular proliferation was minimal, whether the antibody was immobilized alone at 30 μg/ml, or immobilized together with control antibody L20 at concentrations of 10 μg/ml anti-CD3 plus 20 μg/ml L20 (FIG. 4). However, when anti-CD28 mAb was immobilized with anti-CD3, an increase in proliferation of both CD4⁺ and CD8⁺ T cells was observed, and such effects were not further enhanced by addition of more anti-CD28 mAb (FIG. 4). Similarly, co-immobilized anti-CD2 mAb and anti-CD3 mAb increased the proliferation of CD4⁺ and CD8⁺ T cells above the level induced by anti-CD3 alone. When both anti-CD2 and anti-CD28 were added to anti-CD3 during the antibody immobilization step, there was a further dramatic increase in proliferation of CD4⁺ T cells, whereas proliferation of CD8⁺ cells was not enhanced above that induced by anti-CD3 plus anti-CD28 or by anti-CD3 plus anti-CD2 (FIG. 4). These results show that the combination of co-immobilized anti-CD3, anti-CD28 and anti-CD2 antibodies enhanced proliferation of CD4⁺ T cells over the combination of co-immobilized anti-CD3 and anti-CD28 or the combination of anti-CD3 and anti-CD2. In total T cell stimulation, anti-CD3, anti-CD28 and anti-CD2 combination is expected to induce greater amounts of lymphokine production by CD4⁺ T cells, which in turn stimulate greater CD8⁺ T cell activation. In that connection, co-immobilized antibodies stimulate distinct cytokine profiles by activated T cells, depending on which specific combination of three or more antibodies is used. Such activated T cells may be co-cultured with other cell types in vitro such as monocytes or dendritic cells to promote their growth or differentiation in the absence of exogenous cytokines.

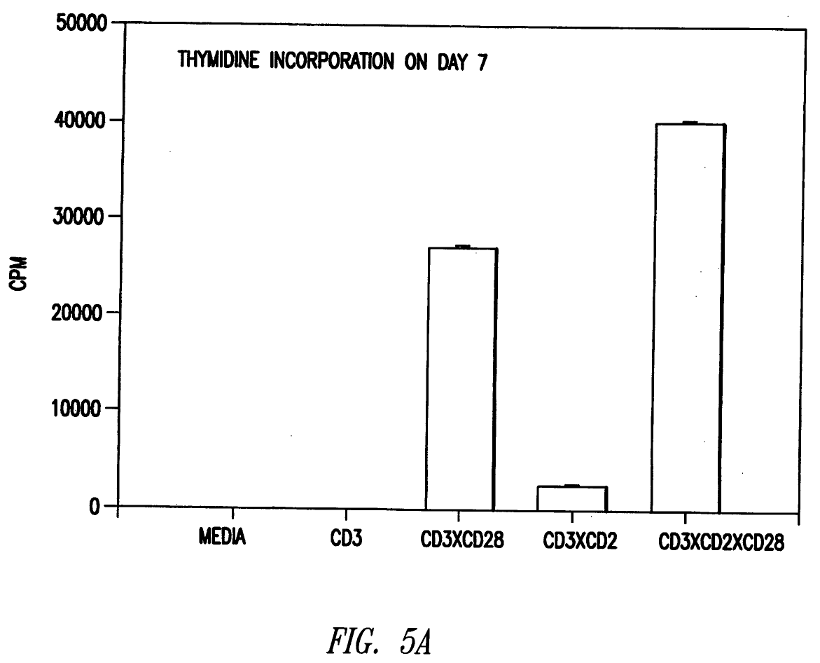

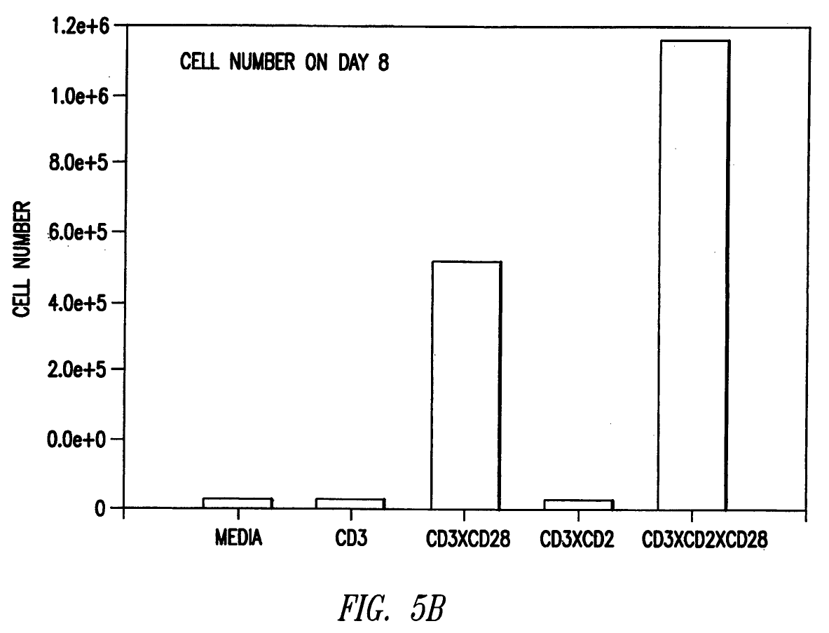

In addition, FIGS. 5A and 5B shows that ³H-thymidine incorporation measurement of T cell proliferation correlated directly with cell growth after stimulation with immobilized antibodies. Proliferation of purified CD4⁺ T cells was measured at day 7 with a 12 hr pulse of ³H-thymidine, while cell number was measured on day 8 by direct cell counting with a hemocytometer. Such findings indicate that measurement of T cell proliferation by ³H-thymidine uptake is directly reflective of the ability of co-immobilized anti-CD2, anti-CD3 and anti-CD28 antibodies to expand T cell numbers in cultures.

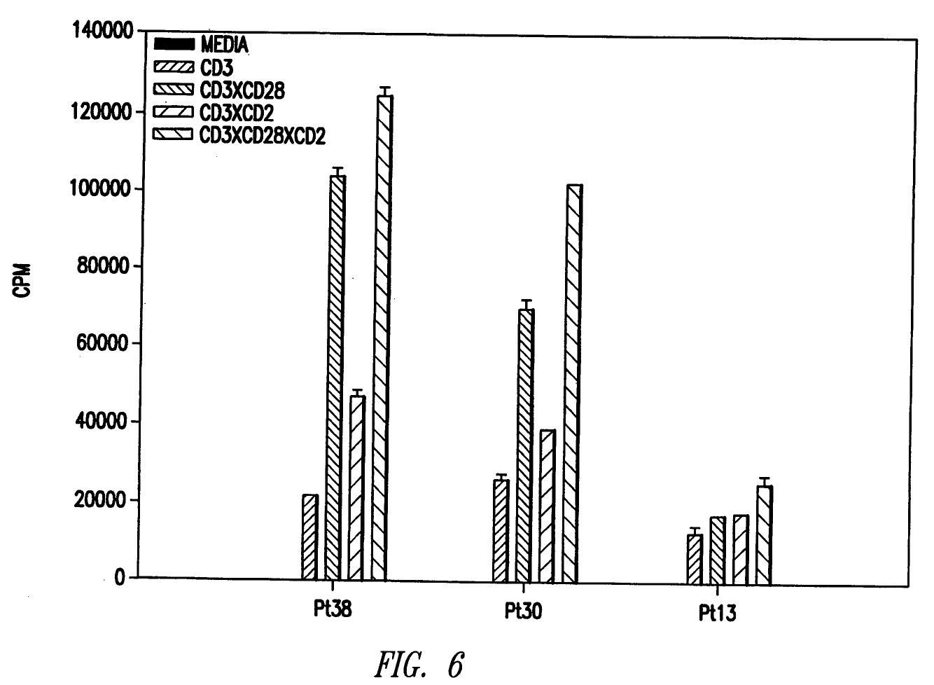

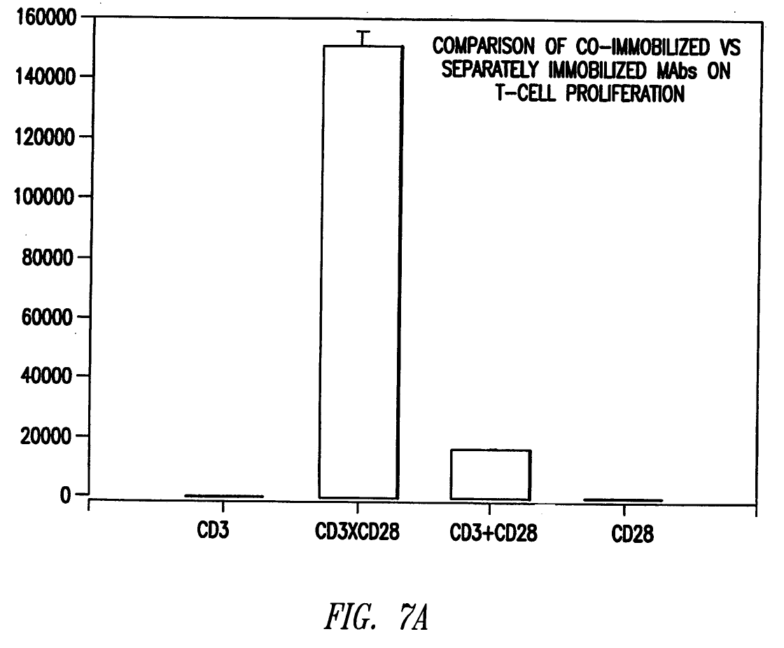

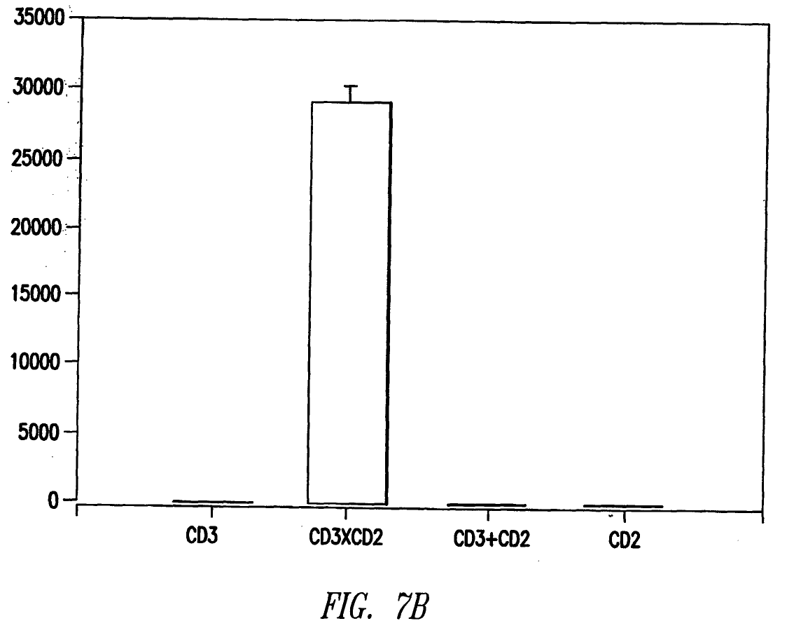

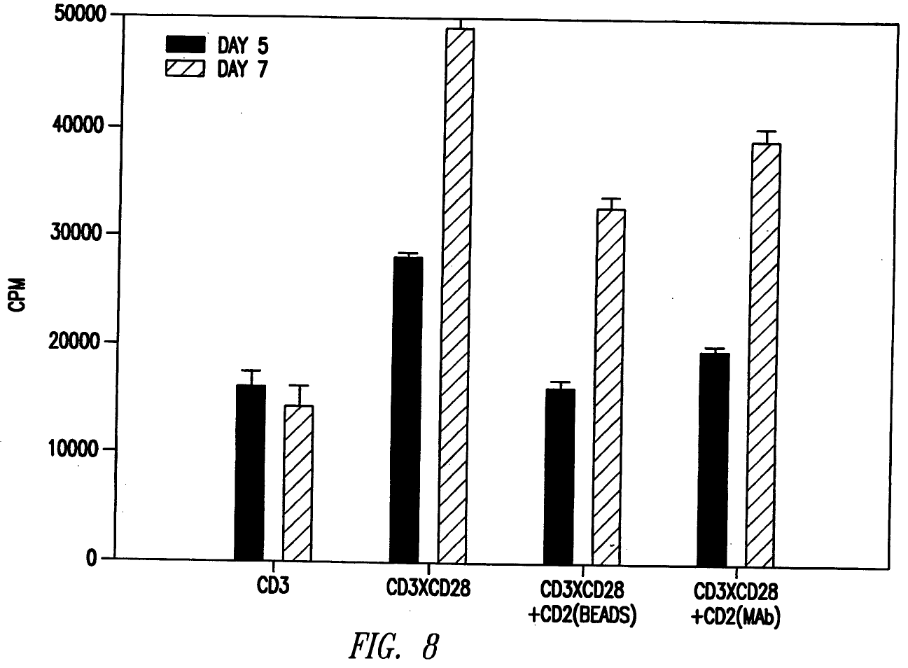

In order to test the ability of the antibodies immobilized on another form of solid support in T cell activation, mAbs were co-immobilized on “DYNAL” beads and incubated with human T cells. FIG. 6 shows that the combination of anti-CD3, anti-CD2 and anti-CD28 antibodies co-immobilized on beads consistently induced the highest level of T cell proliferation from all patients tested as compared to anti-CD3 alone or two antibody combinations. Thus, co-immobilization of antibodies on beads produces superior activation of T cells. Furthermore, FIGS. 7A and 7B demonstrates that co-immobilization of antibodies on the same beads produced higher levels of T cell proliferation than a mixture of beads with separately immobilized antibodies, indicating that aggregation of multiple surface molecules on T cells is achieved optimally by positioning the antibodies in close proximity to each other. In that connection, FIG. 8 shows that anti-CD2 immobilized on separate beads or added in solution inhibited T cell proliferation stimulated by anti-CD3 and anti-CD28 co-immobilized on the same beads.

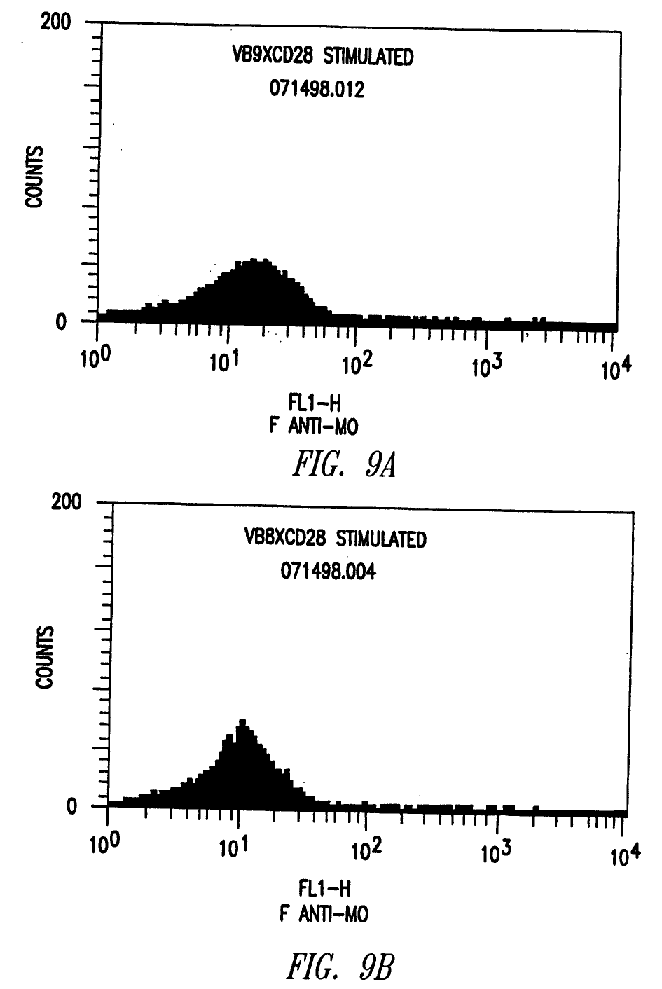

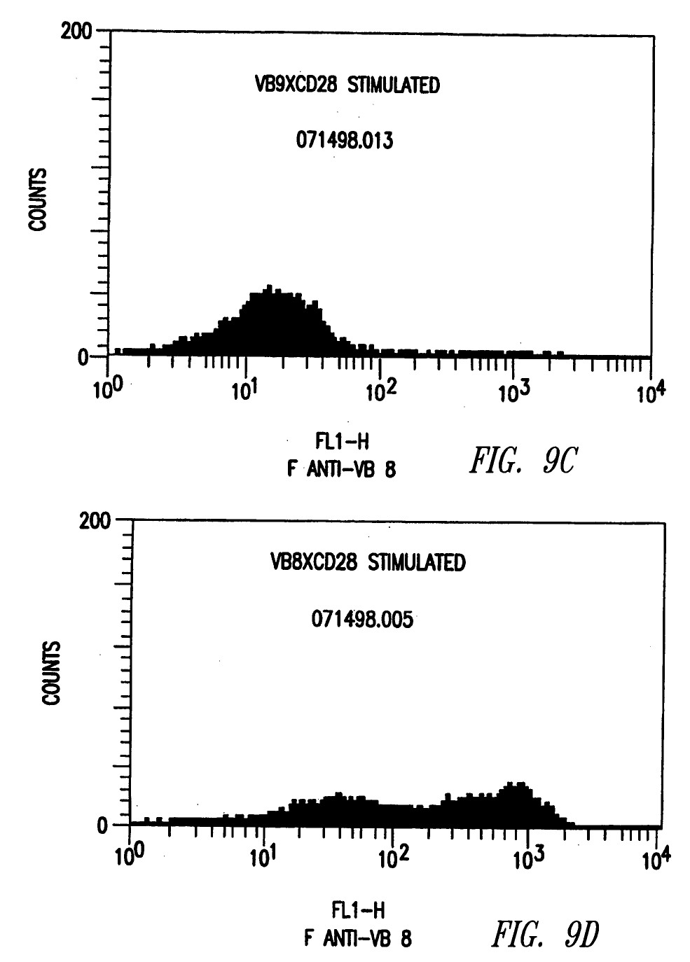

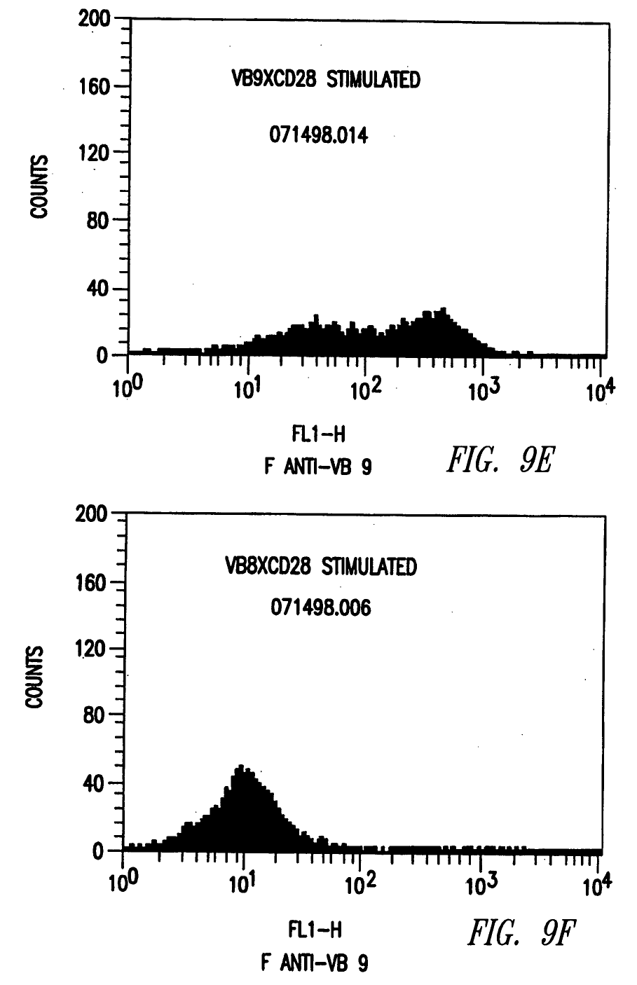

In another experiment, T cells were selectively stimulated by anti-TCR variable region antibodies co-immobilized on culture plates with anti-CD28, followed by analysis of Vβ specificity of the cultured cells. The cells stimulated with co-immobilized anti-TCR Vβ8 and anti-CD28 were 72% positive for expression of Vβ8, but did not express Vβ9, Vβ14, or Vβ20 above the level detected by control anti-mouse IgG second step reagent alone (FIGS. 9B, 9D, and 9F). In contrast, the cells stimulated with co-immobilized anti-TCR Vβ9 and anti-CD28 from the same donor sample did not react with the anti-Vβ8, anti-Vβ14, or anti-Vβ20 antibodies, but reacted significantly (65% positive) with the anti-Vβ9 mAb (FIGS. 9A, 9C and 9E). The cells from this donor analyzed before antibody stimulation showed that expression of each of these Vβ specificities was less that 5%.

These data show that very small subpopulations of T cells can be selectively expanded using mAbs specific for individual TCR Vβ epitopes and an anti-CD28 mAb co-immobilized on a solid surface. Since TCR Vβ usage shows a significant correlation with antigen-specific reactivity of T cells, and TCR Vβ usage can be highly skewed in patients with autoimmune disease and cancer, it is likely that antigen-specific T cells or T cells highly enriched for a specific antigen recognition can be selectively expanded using the appropriate Vβ mAb immobilized with an anti-CD28 mAb. Furthermore, immobilization of a third mAb to an additional T cell antigen, such as CD2, CD150, CD5, or ICOS will further enhance the selective expansion of T cells expressing a specific Vβ. Antibodies to two or more Vβ chains may also be used together with anti-CD28 and additional mAbs to expand T cells expressing the desired Vβ polypeptide chains without expanding the other T cell subsets. Moreover, T cells expressing γδ TCR may also be selectively expanded by a mAb to γδ heterodimer co-immobilized with other antibodies. Any antibody reactive with a component of the TCR/CD3 complex, including any CD3 polypeptide chain or epitopes of the TCR alpha/beta or gamma/delta dimers such as the CDRs may be used for the practice of the invention.

7. Example: Llama B Cells Expressed Cd40 and Produced Heavy Chain-Only Antibodies that Bound Human Cell Surface Antigens

7.1. Materials and Methods

7.1.1. Immunization of Llamas

Llama llama were obtained from JJJ Farms (Redmond, Wash.) and immunized intraperitoneally with human cells in PBS and Freund's complete adjuvant, followed by at least 3 rounds of boosting with the same cells in Freund's incomplete adjuvant. The cell types used for immunization included normal unstimulated or activated human peripheral blood lymphocytes (PBL), T cell lines such as Jurkat and HPB-ALL, B cell lines such as Daudi and Ramos or EBV-transformed line CESS. Llamas were also immunized with 100-500 μg purified fusion proteins in PBS mixed with adjuvant as described above for the cells. Animals were bled 4-7 days after each boost to determine if sera contained antibodies reactive with the target cells. Large bleeds (200 ml) were performed after the third boost or after later boosts, depending on the antibody response of the animal. Animals were bled by venipuncture of the jugular vein and whole blood was treated with citrate anticoagulant.

7.1.2. Preparation of Llama Peripheral Blood

Llama whole blood (200 ml) was centrifuged at 900 rpm for 20 minutes and the upper layer of cells containing peripheral blood mononuclear cells was aspirated to a secondary tube. This fraction was then diluted 1:1 in PBS and 30 ml were loaded onto 15 ml cushions of Lymphocyte Separation Media (LSM, Organon Teknika). Buffy coats were fractionated by centrifugation at 2000 rpm for 20 minutes in a Sorvall tabletop centrifuge and isolated by aspiration from the serum/LSM interface. Cells were washed three times in PBS or serum free RPMI, spun at 1200-1400 rpm for 10 minutes, and counted after the final spin. The appropriate number of cells was aliquoted to fresh centrifuge tubes for the final spin. The final cell pellets were snap frozen without liquid in dry ice-ethanol baths at 10⁸ cells/tube and placed at −70° C. until mRNA isolation. Alternatively, cells were resuspended and cultured overnight in RPMI/10% fetal calf serum at a cell density of 10⁶ cells/ml for use in binding assays or functional studies in vitro. Cells were also frozen in aliquots of 2×10⁷ cells in serum/10% DMSO for use in future functional assays.

7.1.3. Cell Staining and Flow Cytometry

PBL from L. llama were isolated by centrifugation on LSM and the cells were stained with an anti-CD40 mAb, G28-5, (U.S. Pat. No. 5,182,368), an anti-llama immunoglobulin (Ig), and an anti-light chain antibody. The anti-CD40 antibody (G28-5) was labeled with biotin, and its binding was detected with phycoerythrin-conjugated strepavidin. The anti-llama Ig was directly labeled with fluorescein. The anti-light chain staining was performed using fluorescein-conjugated anti-human kappa plus anti-human lambda reagents from Caltag (Burlingame, Calif.). Cell staining was analyzed by a FACSCAN flow cytometer.

7.1.4. Proliferation of Llama Lymphocytes

PBL from L. llama were isolated by centrifugation on LSM. The lymphocytes were stimulated with phorbol-12-myristic acid (PMA) (10 ng/ml), an anti-CD40 mAb (G28-5 at 1 μg/ml), CD86-expressing Chinese hamster ovary (CHO) cells, control CHO cells or combinations of the aforementioned reagents. CHO cells were irradiated prior to the assay to prevent CHO cell proliferation. Lymphocyte proliferation was measured in quadruplicate wells of a microtiter plate containing 50,000 lymphocytes each by incorporation of ³H-thymidine during the last 12 hr of a three day culture period. Means are shown from lymphocyte proliferation results from two different llamas.

7.1.5. Purification of Llama Antibodies

Serum from a llama immunized with multiple injections of Jurkat T cells was fractionated by a multi-step procedure into conventional and heavy chain-only IgG isotypes. Serum was first bound to Protein A, eluted, and then separated by DEAE ion exchange chromatography. The Protein A eluate was separately fractionated by binding to Protein G, followed by elution at pH 2.7 or at pH 3.5. Fractions were analyzed by SDS-PAGE after reduction.

7.2. Results

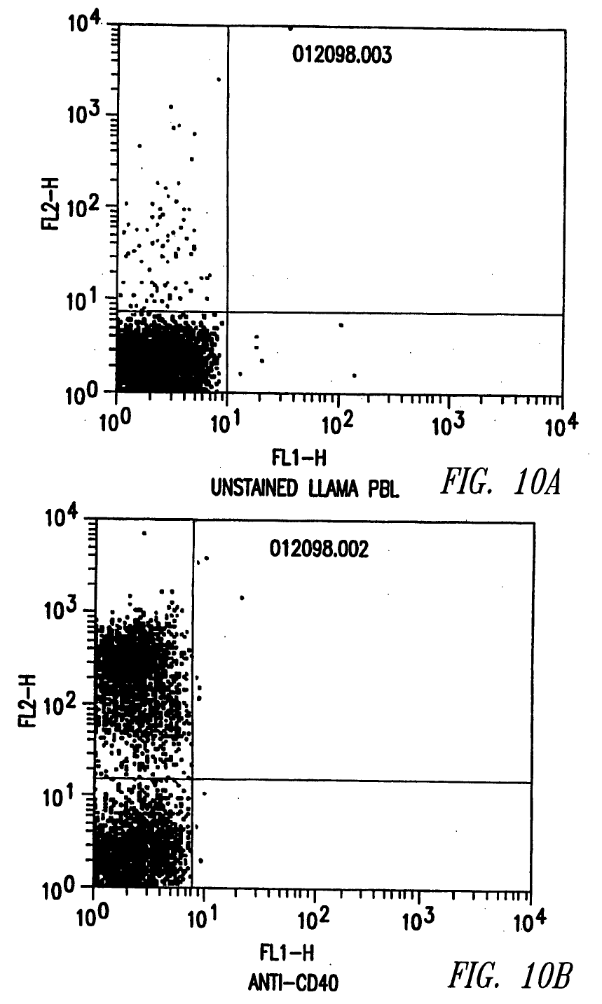

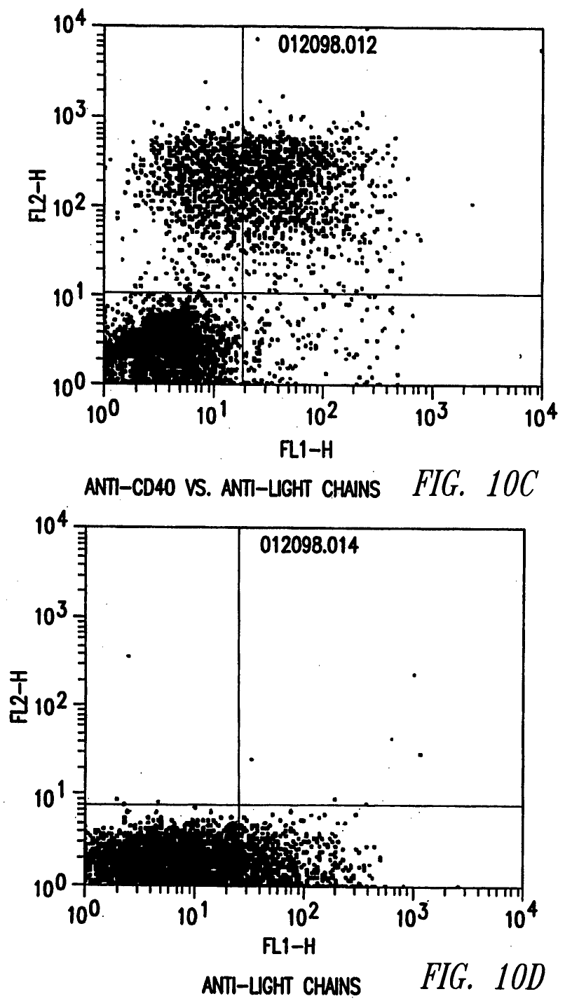

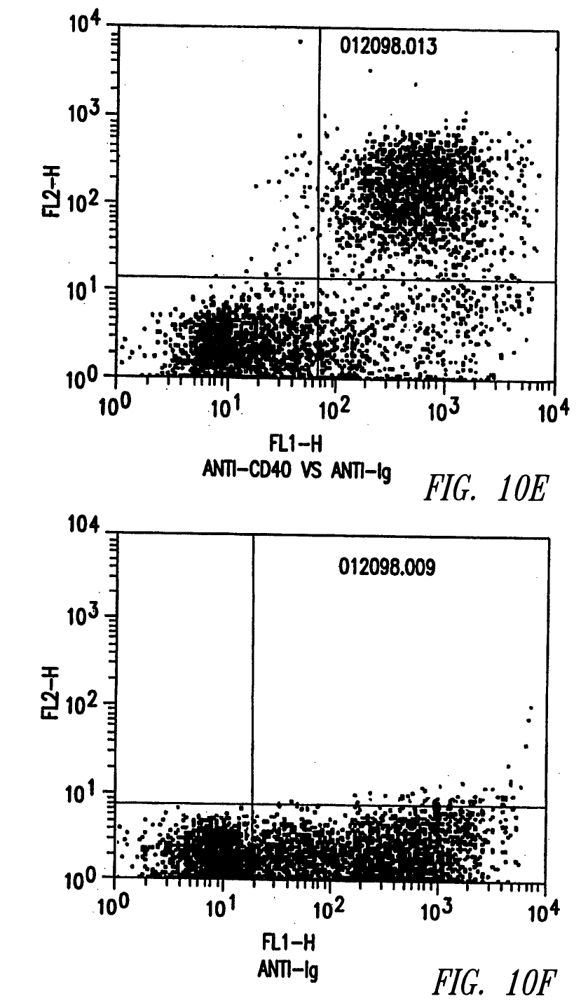

Isolated llama PBL were reacted with anti-CD40 and anti-Ig or anti-light chain antibodies, and analyzed by flow cytometry. FIGS. 10A and 10B shows that a population of llama peripheral blood cells reacted with an anti-human CD40⁺ antibody. Two color staining further demonstrates that all CD40⁺ cells expressed surface Ig, indicating that these cells were antibody-producing B cells (FIGS. 10E and 10F). However, only a portion of the CD40⁺ cells expressed detectable light chain (FIGS. 10C and 10D). These results indicate that llama B cells express conventional antibodies composed of heavy and light chains, and heavy chain-only antibodies devoid of light chains. Thus, llama B cells expressing heavy chain-only antibodies can be separated from other B cells by their reactivity with anti-CD40 and lack of reactivity with anti-light chain reagents.

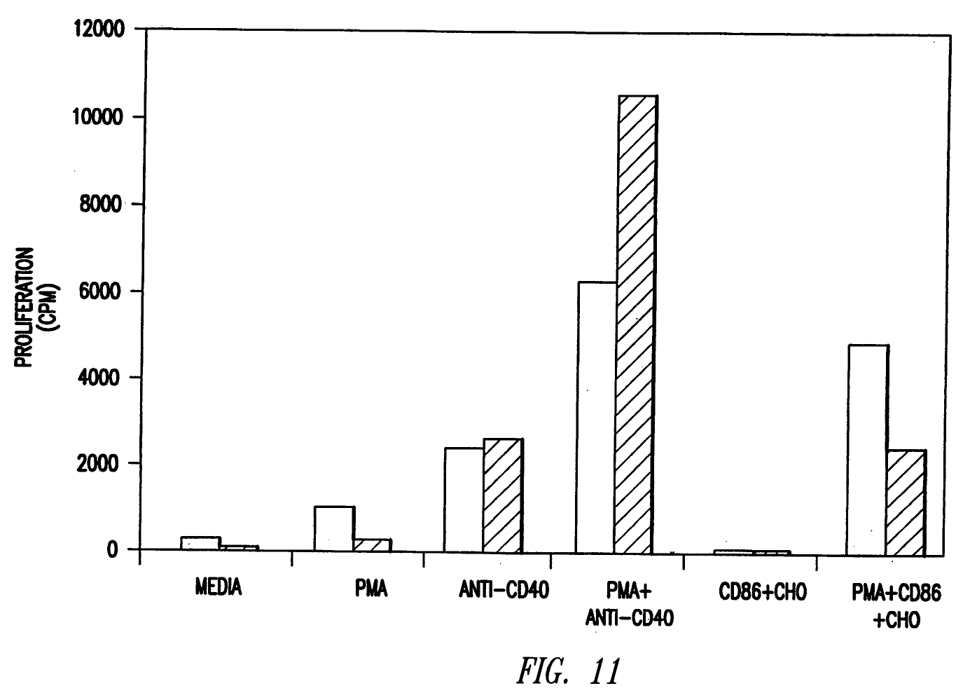

PBL from two llamas were isolated and stimulated with different reagents, followed by measurement of cellular proliferation. Anti-CD40 antibody stimulated llama B cell proliferation, which was further enhanced by PMA (FIG. 11). While CD86 (or B7.2)-expressing CHO cells alone did not induce L. llama B cell proliferation, its combined use with PMA induced significant proliferation (FIG. 11). CD40 stimulation may also induce llama B cell differentiation and Ig affinity maturation in culture. Therefore, CD40 stimulation may be used to selectively expand llama B cells producing heavy chain-only antibodies to facilitate the isolation of these antibodies and their specific V_{HH} regions. In addition, an anti-CD40 antibody may be injected into llamas to stimulate B cells in vivo in order to enhance the number of B cells producing V_{HH}. Cells expressing specific variable regions may be isolated by a variety of methods, including resetting with specific antigen bound to red blood cells.

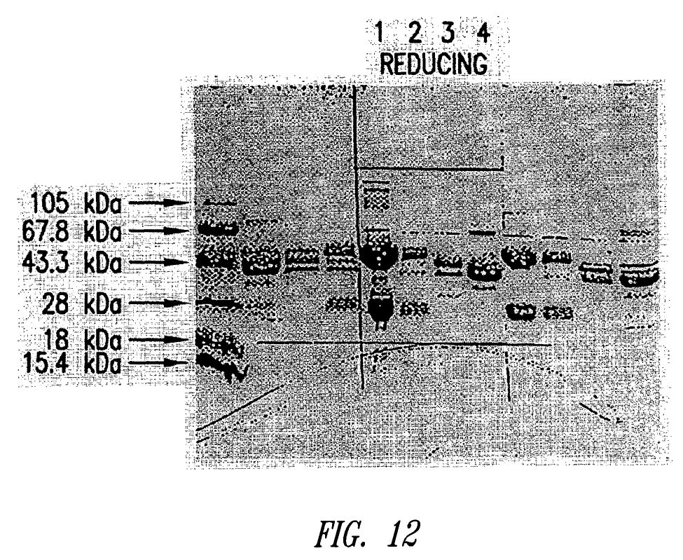

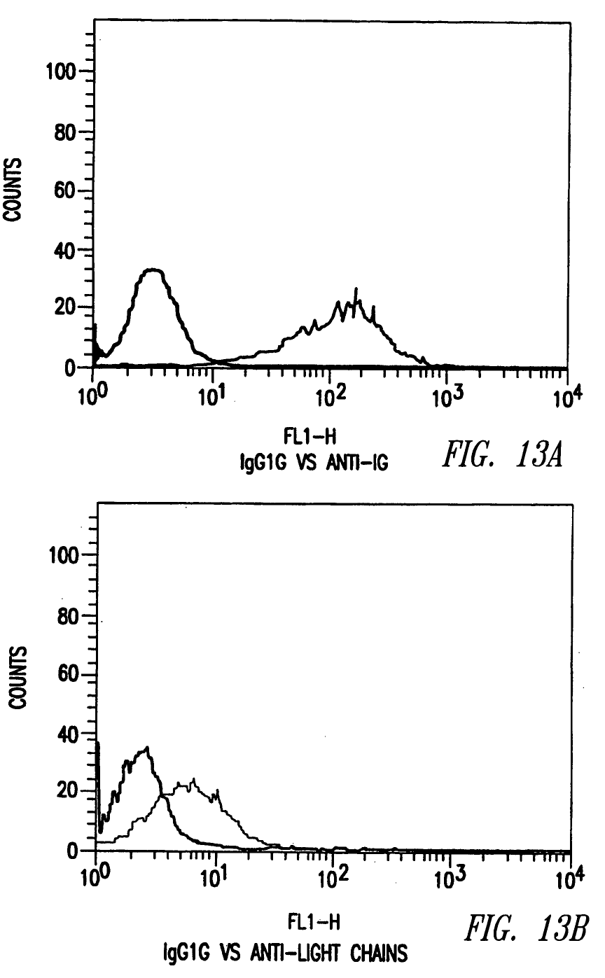

A llama was immunized with human T cells and its serum was fractionated to separate heavy chain-only antibodies from conventional antibodies composed of heavy and light chains. The purified antibody fractions were analyzed by SDS-PAGE. FIG. 12 shows purified Ig isotypes, including IgG1 D (DEAE flowthrough in lane 1), IgG1 G (Protein G, pH 2.7 elution in lane 2), IgG2+IgG3 (Protein G, pH 3.5 elution in lane 3), and IgG3 (Protein G flowthrough in lane 4). The IgG2 and IgG3 isotypes (lanes 3 and 4) contained a heavy chain band without detectable light chain.

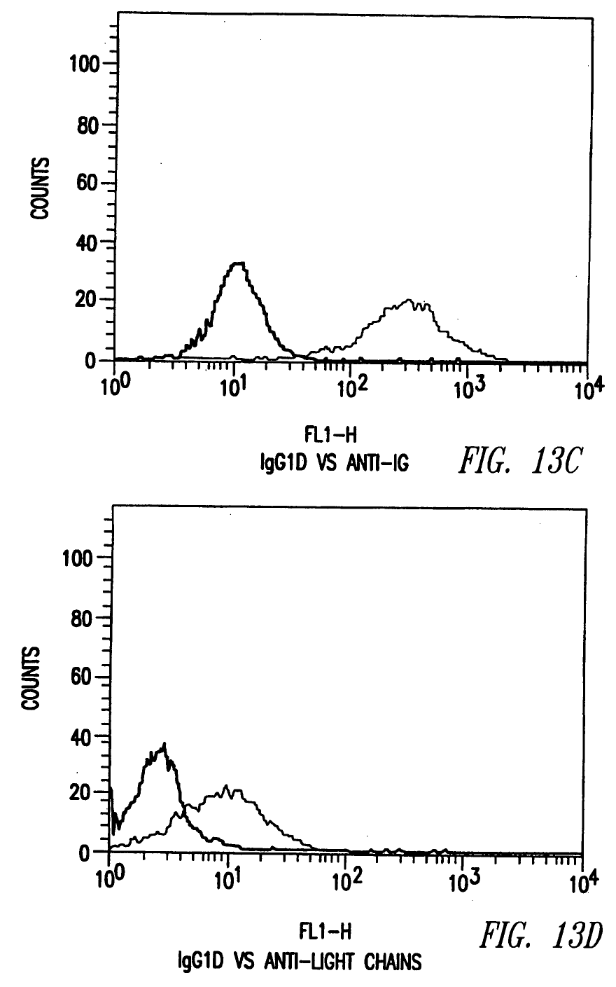

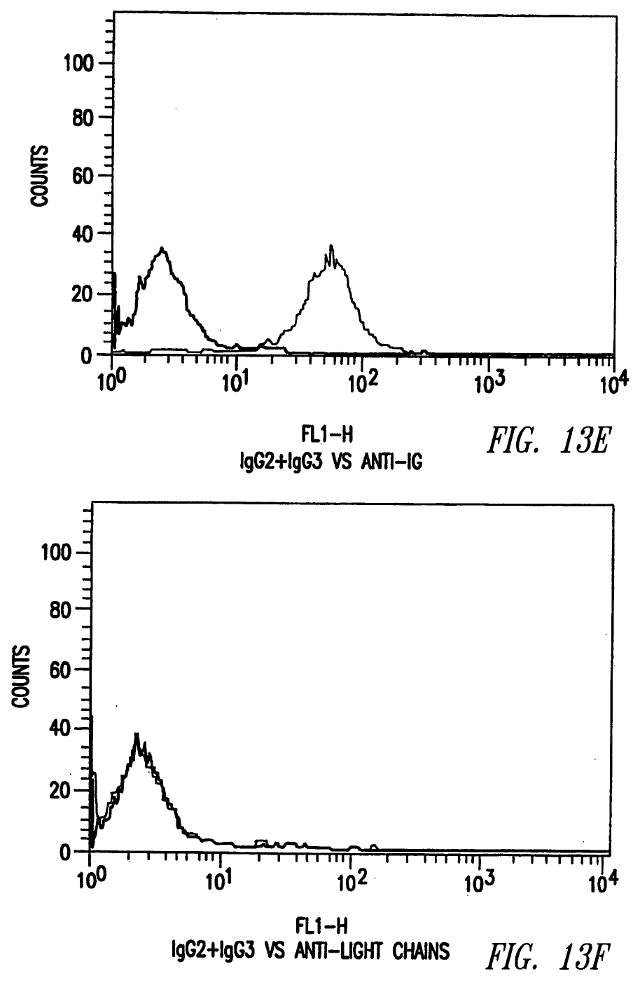

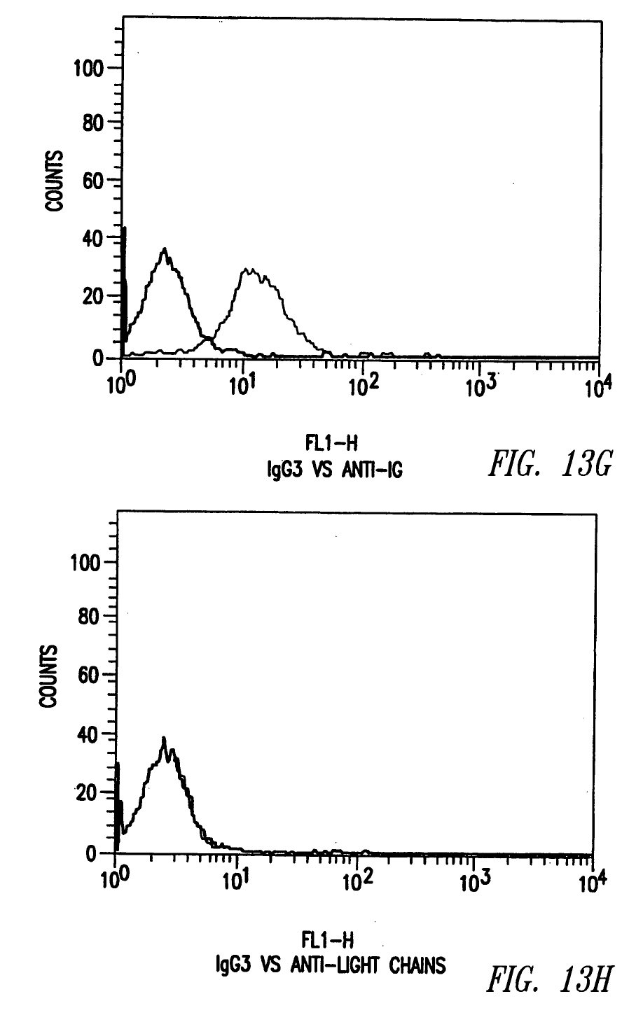

The heavy chain-only antibodies (IgG2+IgG3, and IgG3 fractions) were incubated with Jurkat T cells for detection of antibody binding to cell surface antigens. Specific binding was detected using a fluorescein-conjugated anti-llama Ig or anti-light chain second step reagent, followed by analysis with a flow cytometer (FIG. 13A-13H). Negative controls were purified IgG isotypes at the same concentrations from an unimmunized llama. While the anti-light chain reagent detected binding of the IgG1 fractions (FIGS. 13B and 13D) to the Jurkat cells, the IgG2 and IgG3 fractions which did not contain light chains were not detected with the anti-light chain reagent (FIGS. 13F and 13H). However, when Jurkat cells were stained with the heavy chain-only fractions and detected by an anti-Ig second step reagent, antibody binding to Jurkat cell surface antigens was observed (FIGS. 13E and 13G). It is concluded that llama antibodies devoid of light chain were generated against human cell surface antigens.

8. Example: Construction of L. llama V_{HH} Libraries and Characterization of Llama V_{HH} Sequences

8.1. Materials and Methods

8.1.1. Isolation of llama mRNA

Llama PBL mRNA was prepared by a modification of the guanidinium-thiocyanate acid-phenol procedure of Chomczynski and Sacchi (1987, Anal. Biochem. 162:156-159). For 10⁸ cells, 5-10 ml denaturing/lysis solution was added to prepare RNA. PolyA RNA was isolated using oligo dT cellulose, washed in 75% ethanol/DEPC treated water, recentrifuged, and resuspended in DEPC treated water.

8.1.2. Reverse Transcription-Polymerase Chain Reaction (RT-PCR)

cDNA was generated by random hexamer primed reverse transcription reactions using Superscript II reverse transcriptase (GIBCO-BRL). PCR reactions were performed using the following primer set: The forward primer was LVH5′-1, one of a battery of 20-mers designed from amino-terminal sequencing of the V_{HH} protein, with the sequence 5′CTC GTG GAR TCT GGA GGA GG3′ (SEQ ID No:47), while the reverse primer used was LVH3RS, a 44-mer designed from previously determined, existing camel and human V_{H} sequences. The sequence 5′CGT CAT GTC GAC GGA TCC AAG CTT TGA GGA GAC GGT GACYTG GG3′ (SEQ ID NO:48) annealed at the 3′ end of the V_{H} domain. PCR products were electrophoresed on a 6% acrylamide/0.5×TBE gel, and the bands visualized after ethidium bromide staining. DNA bands were isolated from 2% NuSieve GTG gels (FMC) and purified using Qiaex beads (QIAGEN) according to manufacturer's instructions. Purified DNA after PCR was ligated into the pT-Adv plasmid vector (Clontech, Palo Alto, Calif.), and transformed into E. coli TOP10F′ (Clontech). Once a representative sample of V_{H} and V_{HH} sequences was determined, new primers were designed to select for amplification of V_{HH}-containing fragments with a fragment length distinct from V_{H}-containing fragments based on the absence of the CH1 domain in V_{HH} fragments. These fragments were then purified, cloned into the phage display vector XPDNT, and used as template in generating libraries of llama variable regions containing mostly V_{HH} sequences.

Additional methods for the cloning of llama V_{HH} region sequences are as follows. Llama IgG₂-specific V_{HH} regions were cloned from cDNA prepared from llama PBL and amplified by PCR using a human Vh1 family-specific 5′ primer and a 3′ llama IgG₂ hinge region primer. The sequences of these primers were AGGTGCAGCTGGTGCAGTCTGG (SEQ ID NO: 49) and GGTTGTGGTTTTGGTGTCTTG (SEQ ID NO: 50), respectively.

In addition, llama IgG₂-specific V_{HH} regions were cloned from cDNA prepared from llama PBL and amplified by PCR using a human Vh2 family-specific 5′ primer with a 3′ llama IgG₂ hinge region primer. The sequences of these primers were CAGGTCAACTTAAAGGGAGTCTGG (SEQ ID NO: 51) and GGTTGTGGTTTTGGTGTCTTG (SEQ ID NO: 50), respectively.

Llama IgG₂-specific V_{HH} regions were also cloned from cDNA prepared from llama PBL and amplified by PCR using a human Vh4 family-specific 5′primer with a 3′ llama IgG₂ hinge region primer. The sequences of these primers were AGGTGCAGCTGCAGGAGTCGG (SEQ ID NO: 52) and GGTTGTGGTTTTGGTGTCTTG (SEQ ID NO: 50), respectively.

Llama V_{HH} sequences from the amplifications were pooled and digested with SacI and BamHI, then inserted into the modified phage display vector XPDNT, creating gene III fusion cassettes. The V_{HH} library was transformed into E. coli XL1BLUE bacteria by electroporation and plated to large NUNC bioassay dishes containing SB/amp/tet media. Platings on serially diluted samples were also performed at this step to estimate transformation efficiency. Libraries were scraped into SB/amp/tet containing 20% glycerol and frozen in 1-2 ml aliquots at −70° C. Libraries were amplified in liquid 2XYT/amp/tet+glucose at 37° C. for several hours, then infected with helper phage, plated to determine phage titer, and grown under selective conditions in media lacking glucose at 30° C. overnight. The amplified phage were isolated from these cultures by centrifugation to pellet bacteria, followed by PEG precipitation of culture supernatants, and a second centrifugation to recover phage precipitates. A small aliquot of unprecipitated culture supernatant was also harvested prior to the addition of PEG/NaCl. Precipitates were resuspended in 1/100 volume PBS/1% BSA and spun for several minutes at 2000-5000 RCF to pellet insoluble material. Phage stocks or supernatants were preblocked by incubation in 10% nonfat milk/PBS for 1 hour on ice prior to panning against preblocked human antigen or cells. Many rounds of panning were precleared with untransfected or normal human cells or with irrelevant −Ig fusion protein to reduce the frequency of nonspecific binders. Preclearing and panning were performed by coincubating the blocked phage with antigen or cells for 1 hour on ice and centrifugation to pellet bound phage. For panning with −Ig fusion protein antigens, protein A sepharose was used to capture phage-antigen complexes prior to centrifugation. Bound cells or protein A sepharose were washed at least 6 times and as many as 12 times in 10% milk/PBS, PBS/1% BSA or PBS/blocker/0.05% Tween prior to elution. Elution of bound phage was performed by incubation in one of several different buffers, and incubation for 10 minutes at room temperature. Elution buffers included 0.1N HCl, pH 2.5 in PBS, 0.1 M citric acid pH 2.8, 0.5% NP-40 in PBS, or 100 MM triethylamine. Cells/sepharose were pelleted and the supernatant containing eluted phage aliquoted to fresh tubes. Eluates were neutralized in 1M Tris, pH 9.5, prior to infection of logarithmic XL1BLUE cells. After infection, aliquots were taken to determine eluted phage titers. Random clones from these platings were then amplified to determine insert frequency and DNA sequence at each round of panning. Llama V_{HH} sequences were determined from the initial library and after each round of panning from random clones.

8.1.3. Phage Display Vector

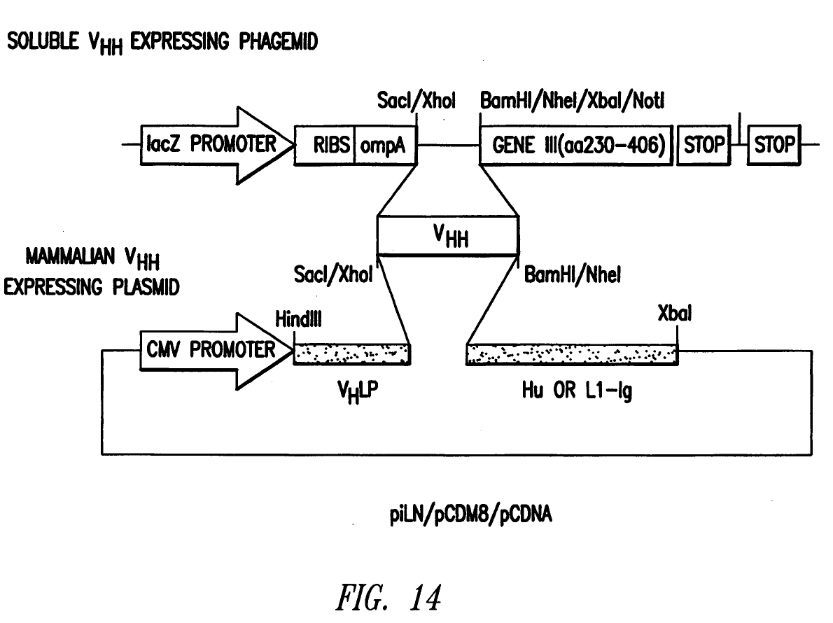

A phage display vector was constructed which created a hybrid fusion protein encoding llama immunoglobulin V_{HH} domains specific for human antigens attached to a truncated version of bacteriophage M13 coat protein III (FIG. 14). The phagemid vector contained a pUC vector backbone, and several M13 phage derived sequences for expression of gene III fusion proteins and packaging of the phagemid after coinfection with helper phage. The vector was constructed in two forms which differed by the manner in which the fusion between the two protein domains was achieved. The first form included a his6 tag between the two domains as a potential tool for purification and detection of functional fusion proteins. The second form lacked this tag and contained only a single (gly₄ser) subunit between the two cassettes. Both versions of the vector were constructed with the gene III fusion out of frame and nonfunctional unless a V_{HH} was inserted between the leader peptide domain and the gene III domain. All V_{HH} molecules were PCR amplified with SacI-BamHI ends for insertion between the ompA leader peptide (EcoRI-SacI) and the gene III fusion beginning at SpeI. Once V_{HH} cassettes with binding activity for human antigens or cells were detected and isolated, the SacI-BamHI fragments could be directly transferred to a mammalian expression vector with compatible sites. The mammalian vector contained a HindIII-SacI leader peptide and a BamHI-XbaI immunoglobulin domain for expressing human, llama, or mouse Ig fusion proteins. This altered vector permitted rapid shuttling of putative antigen binding V_{HH} into a system more amenable to functional analysis.

Individual phage clones were isolated after 3-5 rounds of panning with target antigens. Eluates from each round of panning were infected into host bacteria and aliquots were plated to LB/amp/tet plates for isolated colonies. Individual clones were inoculated into 2XYT/amp/tet liquid media for several hours, infected with helper phage, and grown under selective conditions overnight at 30° C. Phage supernatants were then prepared by centrifugation to pellet cells and culture supernatants were aliquoted to fresh tubes. Precipitated, concentrated phage (100×) were prepared by PEG precipitation of the culture supernatants and resuspension in PBS/1% BSA.

Experimental phage supernatants, precipitates, or helper phage were preblocked 1:1 with 10% nonfat milk/PBS for 30 minutes on ice. Human PBL or monocytes were counted and resuspended in 5% nonfat milk/PBS and preblocked on ice for 30 minutes. Thereafter, cells were pelleted and resuspended in 5% nonfat milk/PBS, added to preblocked phage in 25 μl per sample, and incubated on ice for 1 hour. Following binding, cells were washed 3 times with alternating 5% milk/PBS and 1% BSA/PBS. Mouse anti-M13 antibody at 10 μg/ml in staining media (2% FBS/RPMI+0.1% sodium azide) was added to cells, 100 μl per sample, and incubated on ice for 1 hour. Cells were washed 3 times as above. FITC-conjugated goat F(ab′)₂ anti-mouse Ig (gamma and light, AMI4408 BioSource Int.) 1:100 in staining media was added to cells, 100 μl per sample, and incubated on ice for 30 minutes. Stained samples were then washed and analyzed by flow cytometry.

8.1.4. Sequencing of DNA Fragments

Subcloned DNA fragments were subjected to cycle sequencing on a PE 2400 thermocycler using a 25 cycle program with a denaturation profile of 96° C. for 10 seconds, annealing at 50° C. for 30 seconds, and extension at 72° C. for 4 minutes. The sequencing primers used were the T7 promoter primer 5′TAA TAC GAC TCA CTA TAG GGA GA3′ (SEQ ID NO: 53) and the M13 reverse sequencing primer 5′AAC AGC TAT GAC CAT G3′ (SEQ ID NO: 54) (Genosys Biotechnologies, The Woodlands, Tex.). Reactions were performed using the Big Dye Terminator Ready Sequencing Mix (PE-Applied Biosystems, Foster City, Calif.) according to manufacturer's instructions. Samples were ethanol precipitated, denatured, and analyzed by capillary electrophoresis on an ABI 310 Genetic Analyzer (PE-Applied Biosystems). Sequence was edited and translated using Sequencher 3.0 (Genecodes).

8.2. Results

Llamas were immunized with human lymphocytes or fusion proteins for the generation of antibody responses against lymphocyte surface antigens as described in Section 7.1.1, supra. After immunization, llama PBL were prepared and V_{HH}-containing DNA fragments were obtained by RT/PCR for the construction of V_{HH} libraries.

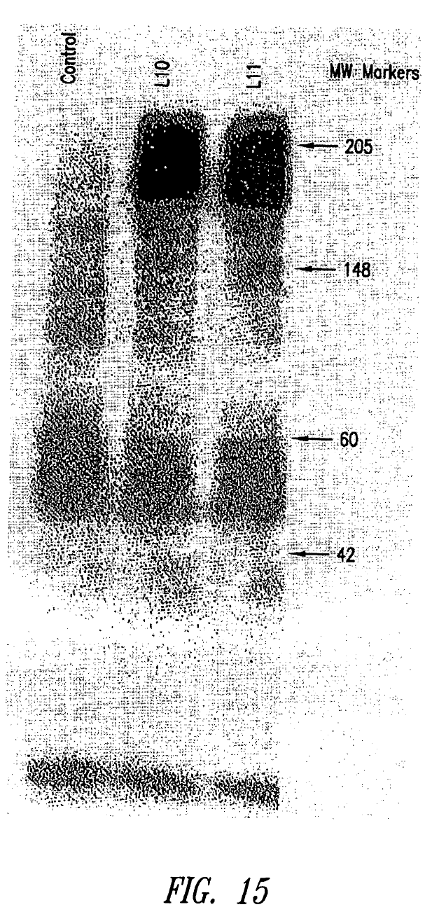

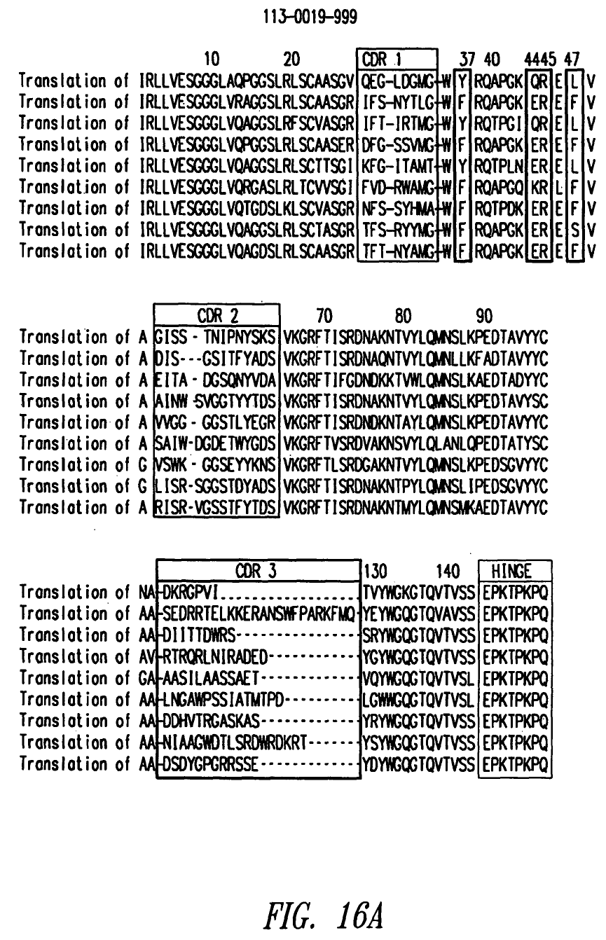

A phage display vector was constructed for the cloning of cell-binding V_{HH} sequences from llamas immunized with human lymphocytes (FIG. 14 and Section 8.1.3., infra). Table I shows several isolated phage clones, each of which exhibited a characteristic pattern of binding to different human cell types. Subsequent sequence analysis verified that each clone encoded a unique V_{HH}. In addition, two V_{HH} clones, L10 and L11, were isolated which reacted with a high molecular weight glycoprotein of 150-200K Da antigen expressed on CHO cells (FIG. 15). Binding of these clones to the target antigen was completely abrogated when CHO cells were pre-treated with trypsin. V_{HH} binding was only partially reduced following treatment of cells with neuraminidase or other endoglycosidases. Thus, the V_{HH} clones reacted with a glycoprotein expressed on the surface of CHO cells.

A number of llama V_{HH} DNA clones were isolated, sequenced and translated. As the phage clones were selected by several rounds of panning on dishes containing an

TABLE I
Binding Patterns of Phage Clones of V_{HH} to Different Cell Types

| | CCA3 | CCA6 | CCA13 | CCA16 | CCA17 | CNP5 | CNP6 | CNP8 | CNP15 |
|---|---|---|---|---|---|---|---|---|---|
| lymphocytes | 29%+ | 36%+ | 11%+ | 26%+ | 34%+ | 20%+ | 13%+ | 26%+ | 12%+ |
| monocytes | + | + | − | + | + | − | − | + | − |
| T51 | ++ | ++ | + | +++ | ++ | ++ | + | + | + |
| 616 | ++ | ++ | + | +++ | ++ | ++ | + | + | + |
| CESS | + | + | − | + | + | + | − | + | − |

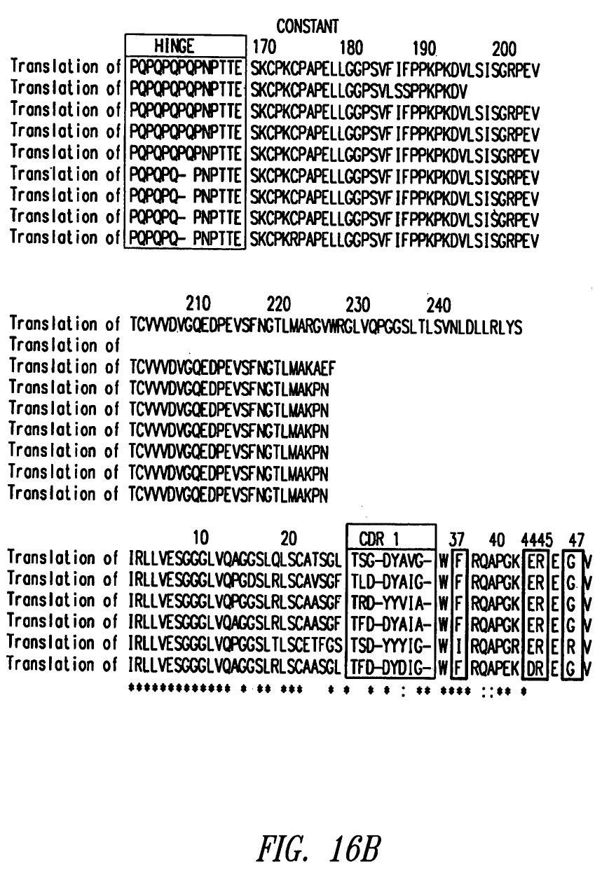

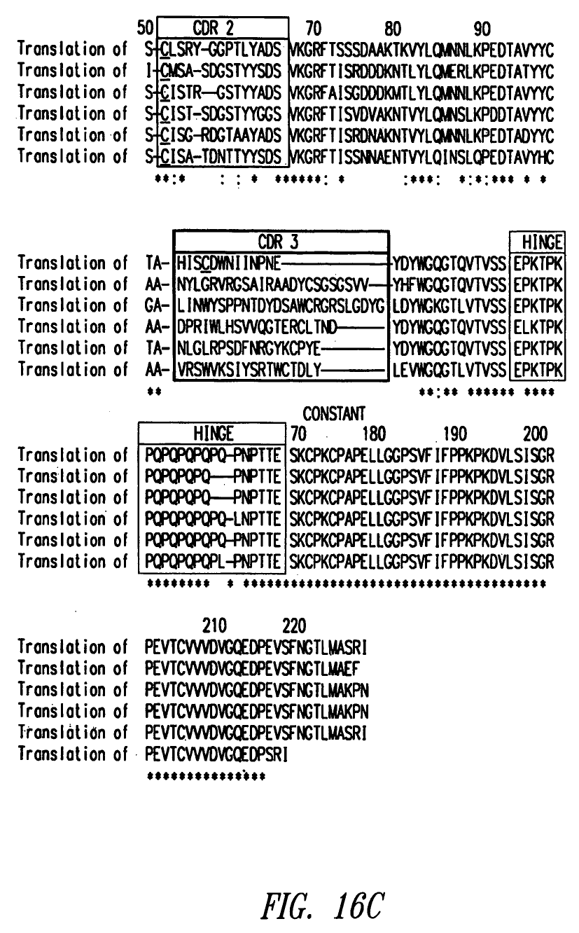

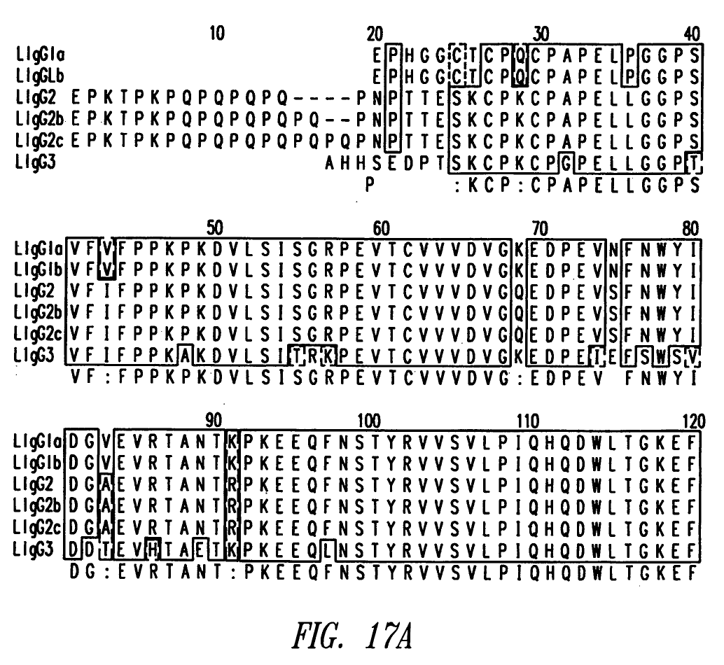

antigen or antigen-expressing cells, sequence diversity of the clones was reduced after five rounds of panning. The resulting protein sequences of the V_{HH} were aligned to identify sequence motifs present in this family of antibody variable regions from L. llama. Sequence alignment revealed two subclasses of V_{HH} sequences in L. llama, which are referred to herein as hybrid (SEQ ID NOS:1-9) and complete (SEQ ID NOS:10-15) V_{HH} sequences. Neither subclass contains a CH1 domain of conventional heavy chains, and thus both are expressed as V_{HH} domains fused directly to the hinge-CH2-CH3 domains of an antibody. The hypervariable domains CDR1, CDR2 and CDR3 present in most antibody variable regions are seen in both types of V_{HH} molecule (FIGS. 16A and 16B). The CDR3 sequence in L. llama V_{HH} domains is longer on average than most CDR3 domains of conventional antibodies composed of heavy and light chains, with the longest CDR3 shown in FIG. 16B containing 26 amino acid residues. It was previously reported that the CDR3 and CDR2 (or occasionally the CDR1 domain) domains in camels usually contained a cysteine residue which was hypothesized to be involved in the formation of a disulfide linkage between the two CDR domains (Muyldermans et al., 1994, Prot. Engin. 7:1129-1135). While this residue is present in the CDRs of the molecules classified as complete V_{HH} (FIG. 16B), the sequences of the hybrid subclass do not contain a cysteine in the CDR1, CDR2, or CDR3 domain (FIG. 16A). Therefore, this class of V_{HH} molecules from L. llama are unique and distinct from dromedary species. CDRs derived from this subclass may be superior in stability as they function independently without disulfide linkages between them.

Based on the aforementioned sequence information, several amino acid residues in the variable regions were identified as important in formation of the V_{L}-V_{H} interface, including residues 11, 37, 44, 45, and 47 (Table II). Amino acid residues in four positions were reported to be hydrophilic residues in camel antibodies. Changes in these residues are also found in llama V_{HH} domain, and they may alter the solubility of the unpaired polypeptides. However, although the leucine at residue 11 is usually substituted with a serine in camels, the majority of L. llama sequences contain a leucine at this position. Subsequent clones showed that llama sequences occasionally contained lysine, serine, valine, threonine or glutamic acid at this position.

The amino acids at positions 44, 45, and 47 of camel antibodies have been reported to contain hydrophilic amino acid substitutions for the usual hydrophobic residues observed in conventional V_{H} domains (44-Gly, 45-Leu, and 47-Trp, respectively). There are some exceptions to this general observation of hydrophilic substitution in the hybrid subclass of V_{HH} domains. Residue 45 for all camel and llama species is the only position which contains an invariant hydrophilic Arg residue substituted for the Leu residue found in conventional V_{H} domains. Certain rare sequences containing isoleucine at this position have been observed. Residue 47 (Trp) is more variable, encoding a Gly or Arg in the L. llama complete V_{HH} sequences, but encoding the hydrophobic residues Leu or Phe in the hybrid V_{HH} sequences. Subsequent clones have been found to contain tryptophan, isoleucine, serine or alanine as well. Residue 44 (Gly) is also more variable, substituting Glu or Asp for Gly in the complete V_{HH} subclass, while Glu, Lys, and Gln occur at this position in the hybrid group. A clone containing threonine at position 44 has also been isolated.

In summary, the hybrid subclass family of V_{HH} sequences possess the following characteristics:

1. These variable region polypeptides are derived from antibodies devoid of light chains, which contain no CH1 domains.

2. They do not contain a disulfide linkage between the CDRs.

3. The amino acid residue at position 11 is usually a leucine instead of serine.

TABLE II
Unique Amino Acid Residues in Llama Antibody
Variable Regions

| | amino acid position | | | | |
|---|---|---|---|---|---|
| | 11 | 37 | 44 | 45 | 47 |
| Mouse | L | V | G | Q | C |
| | L | V | G | L | W |
| Previously | S | Y | E | R | F |
| Reported Camel | S | F | E | R | G |
| Previously | S | F | E | R | G |
| Reported Llama | L | F | E | R | G |
| New V_{HH} Llama | S | F | E | R | G |
| clones | S | F | D | R | G |
| | K | F | E | R | G |
| | L | F | E | R | G |
| | L | F | E | R | F |
| | L | F | E | R | S |
| | L | F | E | R | A |
| | L | F | D | R | G |
| | L | F | D | R | F |
| | L | F | K | R | F |
| | L | F | K | R | P |
| | L | F | Q | R | L |
| | L | Y | E | R | L |
| | L | Y | T | R | L |
| | L | Y | Q | R | L |
| | L | Y | A | R | F |
| | L | Y | E | R | I |
| | L | Y | E | R | G |
| | L | L | E | R | G |
| | L | V | E | R | G |
| | L | Y | K | R | R |
| | L | V | G | L | W |
| | L | V | E | L | W |
| | L | V | E | I | W |
| | L | I | E | R | R |
| | L | I | D | R | R |
| | L | I | D | R | L |
| | L | I | E | I | G |
| | L | A | P | L | W |
| | S | I | E | R | F |
| | S | Y | Q | R | W |
| | S | Y | Q | R | F |
| | V | F | E | R | F |
| | T | F | E | R | Y |
| | E | Y | L | R | M |

9. Example: Cloning of Llama Immunoglobulin Constant Region Coding Sequences

9.1. Llama Serum Assay

To test the serum reactivity against antigens expressed as llama IgG fusion proteins, the antigen-llamaIgG fusion proteins were coupled to “DYNAL” beads and incubated with a serum sample from an immunized llama. The antigen-bead complex was then spun out of solution, washed and incubated on ice in 0.1M citric acid pH 2.3 to remove any antigen-reactive proteins bound during the serum incubation. The antigen-bead complex was again spun out of solution and the supernatant was neutralized in one half volume 0.1M Tris pH 9.5. An equal volume of SDS-PAGE sample buffer containing 2-mercaptoethanol as a reducing agent was added to the neutralized proteins and heated at 100° C. for 5 minutes. The sample was then run on a 10% Tris-glycine polyacrylamide gel and transferred to a nitrocellulose filter. The filter was blocked in PBS+5% non-fat dry milk+0.01% NP 40, then incubated in blocking buffer+1:5000 dilution goat anti-camelid IgG-HRP conjugate. The filter was then washed in PBS+0.01% NP 40 and incubated in ECL reagent. Proteins were visualized by autoradiography.

9.2. Results

Llama constant region coding sequences were cloned using a series of oligonucleotide primers. RNA from llama PBL was isolated and cDNA prepared using random primers or oligo dT. Specific primers designed to amplify the constant domains of the antibody heavy chain were then used to PCR the different llama isotypes.

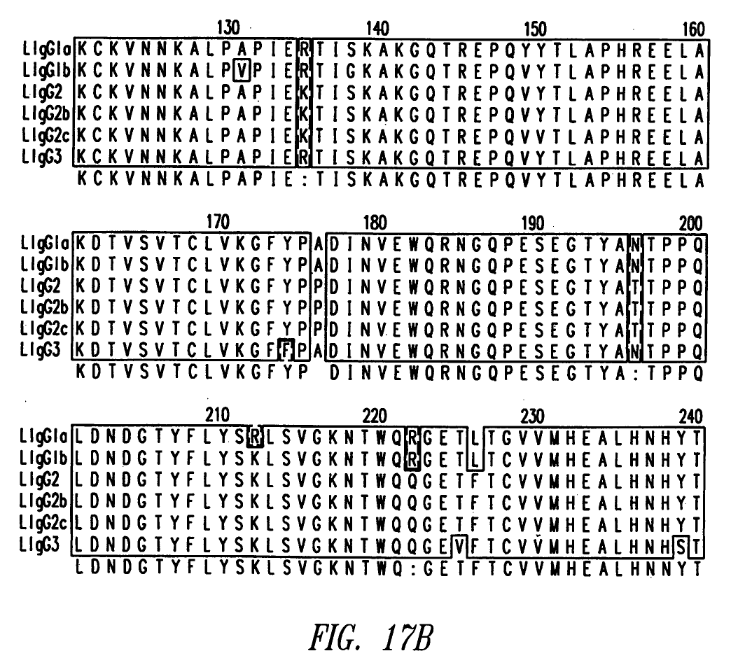

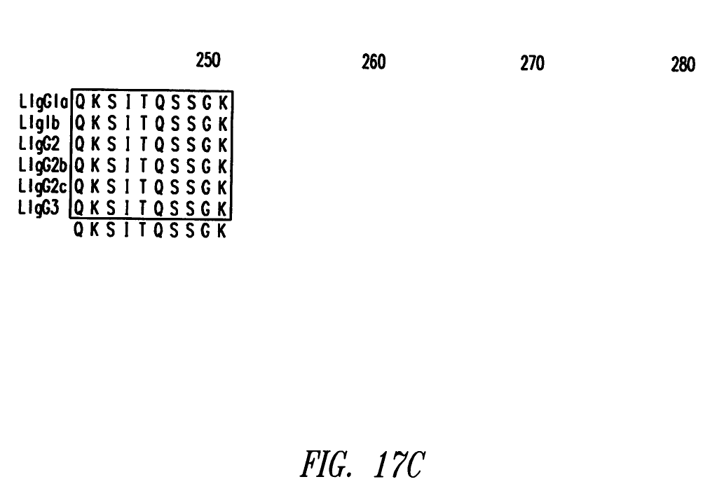

Alignment of the cloned constant region sequences obtained from llama heavy chain genes is shown in FIG. 17. Only sequences from the hinge region to the CH3 domain were compared, since IgG₂ and IgG₃ lack CH1 domain. The hinge domains vary most in length and sequence. Other sequence variation is limited to a few residues scattered throughout the molecule.

Llama constant region coding sequences were ligated with various human leukocyte antigen coding sequences for the expression of fusion proteins. Table III shows a number of recombinant fusion proteins between llama constant regions and human lymphocyte surface antigens which retained the surface antigen binding activities. The different hinge regions of llama IgG₁, IgG₂ and IgG₃ allow for the design of different types of fusion proteins, depending on whether the naturally-occurring molecule is a monomer or dimer. Fusion proteins with llama constant regions are particularly useful as immunogens for immunizing llamas because they do not stimulate anti-constant region immune responses, thereby maximizing the antibody response against the non-immunoglobulin portion of the molecule.

In one experiment, a llama was immunized with a human CD40/llama IgG₁ fusion protein at 250 μg in PBS. Pre-immune serum was collected prior to immunization. Serum was also collected from the llama two weeks after the first immunization, followed by a second immunization. Then serum was again collected two weeks later. When the llama serum collected at different time points was analysed by SDS-PAGE, an anti-CD40 IgG₁ response was observed following the first immunization. After the second immunization, anti-CD40 activity was detected in both IgG₁ and IgG2 fractions. Thus, the CD40/Ig fusion protein was a potent immunogen in llama; and could be used as a tool for detecting serum reactivity of the host during the course of immunization.

10. Example: Llamalization of Mouse Antibody Variable Regions

10.1. Materials and Methods

10.1.1. Oligonucleotides for Llamalization

A pair of complementary oligonucleotides was designed at the approximate midpoint of an antibody variable region coding sequence. The DNA duplex formed by these annealed oligonucleotides was the starting point for constructing the rest of a V-region using overlapping single stranded primers which extended the length of the starting oligonucleotides by 18-24 bases at both ends. Since the DNA was very short at

TABLE III
Recombinant Fusion Proteins Between Llama Ig Constant Regions and Human Leukocyte Antigens

| Fusion Protein | Constant Regions | Expression | Purified by Protein A | Activity |
|---|---|---|---|---|
| human (hu)CD28 | llama(L1)IgG1 (hinge, | Positive by SDS-PAGE | Yes | Positive for binding to |
| | CH2, CH3) | | | CD80⁺CD86⁺ cells |
| huCTLA-4 (CD152) | L1IgG1 (hinge, CH2, | Positive by SDS-PAGE | Yes | Positive for binding to |
| | CH3) | | | CD80⁺CD86⁺ cells |
| huCD40 | L1IgG1 (hinge, CH2, | Positive by SDS-PAGE | Yes | Positive for binding to |
| | CH3) | | | CD154⁺, activated T cells |
| huCD80 | L1IgG2 (hinge, CH2, | Positive by SDS-PAGE | Yes | Positive for binding to |
| | CH3) | | | CD28⁺ cells |
| huCD86 | L1IgG2 (hinge, CH2, | Positive by SDS-PAGE | ? | ? |
| | CH3) | | | |
| huB7-3 | L1IgG2 (hinge, CH2, | Positive by SDS-PAGE | ? | ? |
| | CH3) | | | |
| huCD2 | L1IgG, IgG3 | Negative when fused to | ? | ? |
| | | L1IgG2, others pending? | | |

this stage, cycling times during the PCR were kept very short (10 seconds annealing and 20 seconds extension times for the first six reactions, and increasing to 30 second extension for the remaining reaction sets) and the molar amount of overlapping primer was kept low as well. Stock solutions of each primer pair were prepared with concentrations ranging from 1 μM to 32 μM. These stocks were then diluted 1:20 into the PCR mix and added to the existing reactions for each successive 10 cycle step. With each consecutive amplification step, the molar concentration of newly added primer was increased and the cycling times were adjusted for slightly longer extensions. In this way, the de novo construction of the desired coding sequence proceeded bidirectionally and was terminated by a final PCR that added unique restriction sites to each end of the DNA to facilitate cloning.

Applying this method to mouse antibody 9.3, the 9.3V_{H} molecule was resynthesized by diluting all primers in TE at a final concentration of 64 μM. Primer sets were then prepared by mixing the complementary primer pair together in equimolar amounts as the starting pair. All other primers were combined in pairwise sets that overlapped the previous set in both the 5′ and 3′ direction. These primer pairs were then diluted so that the final concentration of primers ranged from 1 μM to 32 μM in TE. The reaction for the first PCR cycling was prepared as follows: 12 ng primer pair H31-47 (SEQ ID NO:28) and HAS47-31 (SEQ ID NO:31) were added to the reaction mix so that the final concentration was 0.6 ng/μl, followed by the addition of 1 μl of a 1 μM stock of primer pair 2 containing primers H22-36 (SEQ ID NO:27) and H54-40 (SEQ ID NO:34) (final concentration was 50 nM), and 17 μl PCR mixture containing ExTaq (TaKaRa Biomedicals, Siga, Japan) dilution buffer, dNTPs, distilled water and ExTaq DNA polymerase (1 unit) according to manufacturer's instructions. The reaction was incubated for 10 cycles with a denaturation at 94° C. for 30 seconds, annealing at 55° C. for 10 seconds, and extension at 72° C. for 20 seconds. Alternatively, for llamalization of the V_{H}, the first primer pair used was (LV1 and L1HAS) (SEQ ID NOS:29 and 32) or (LV2 and L2HAS) (SEQ ID NOS:30 and 33) and 1 μl of a 1 μM stock of primer pair H22-36 (SEQ ID NO:27) and L1H54-40 (SEQ ID NO:35) (or L2H54-40; SEQ ID NO:36) was added to the first reaction. The second 10 cycle reaction proceeded under the same cycling conditions after addition of 19 μl PCR mix and 1 μl of primer pair H22-36 (SEQ ID NO:27) and H62-49 (SEQ ID NO:37) (2 μM stock). A third 10 cycle PCR was performed after addition of 19 μl PCR mix and 1 μl of primer pair H13-27 (SEQ ID NO:26) and H70-57 (SEQ ID NO:38) (4 μM stock). A fourth round of PCR was performed using the same conditions and 1 μl of primer pair H4-18 (SEQ ID NO:25) and H78-65 (SEQ ID NO:39) (8 μM stock). The fifth round of PCR utilized identical conditions after addition of 19 μl PCR mix and 1 μl of primer pair HRS 1-10 (SEQ ID NO: 24) and H84-73 (SEQ ID NO:40) (16 μM stock). A 20 cycle reaction was performed under identical conditions after addition of 1 μl primer pair HRS 1-10 and H92-81 (SEQ ID NO:41) (32 μM stock). Eight microliters of the PCR were subjected to agarose gel electrophoresis to check for amplification. The rest of the PCR was purified using PCR quick columns (QIAGEN) according to manufacturer's instructions and eluted in 50 μl TE.

New PCR were then set up beginning the whole series of reaction sets over in terms of increasing concentrations of primers and extension time. To 18 μl PCR mix was added 1 μl PCR product eluate and 1 μl primer pair HRS1-10 and H100-87 (SEQ ID NO:42) (1 μM). Reactions were denatured for 1 minute at 94° C., followed by a new 10 cycle program using a 30 second denaturation step at 94° C., a 55° C. annealing step for 10 seconds, and a 72° C. extension step for 25 seconds. The next PCR was performed under identical conditions, but using 19 μl PCR mix plus 1 μl primer pair HRS1-10 and H104-95 (SEQ ID NO:43) (2 μM). The third round of PCR was performed using a 10 cycle program identical to the others except for an increase in the extension time at 72° C. to 30 seconds, addition of 19 μl PCR mix and 1 μl primer pair HRS 1-10 and H111′-100 (SEQ ID NO:44) (4 μM). The fourth round of PCR was performed after addition of 19 μl PCR mix and 1 μl primer pair HRS1-10 and H3RS-104 (SEQ ID NO:46) (8 μM). For llamalization, the primer pair used was HRS1-10 and 93VH3′-BAM (SEQ ID NO:45) (8 μM). The 80 μl PCR reaction was PCR-Quick purified and eluted in 30 μl TE. A final PCR reaction was set up using 0.5 μl of PCR eluate, 5%10×ExTaq buffer, 4 μl 2.5 μM dNTPs, 40 μl dH2O, 1 μl primer pair HRS1-10 and H3RS-104 (or 93VH3-BAM). The reaction conditions included a denaturation step at 94° C. for 60 seconds, a 30 cycle program with denaturation at 94° C. for 30 seconds, annealing at 55° C. for 10 seconds, and extension at 72° C. for 40 seconds, followed by a final extension at 72° C. for 2 minutes, and a hold at 4° C. until recovery. The leader peptide was ultimately attached by repeating two PCR cycles using the subcloned PCR product above as template. The primer pair OKT3/9.3HYB (SEQ ID NO:23) and 93VH3-BAM (or H3RS-104) were included in the first 10 cycle reaction with an extension time of 30 seconds at 72° C. A second 10 cycle PCR was performed by adding the primer pair OKT3VHLP-S (SEQ ID NO:22) and 93VH3-BAM (or H3RS-104) under similar reaction conditions as those described for the initial PCRs, but with the longer extension time. Finally, a 30 cycle PCR was performed on the PCR-quick purified product as template and the last primer pair OKT3VHLP-S and 93VH3-BAM (or H3RS-104) as primers to generate a new V_{H} with the leader peptide from OKT3 V_{H} attached.

10.1.2. Llamalized Antibody Production and FACS Analysis

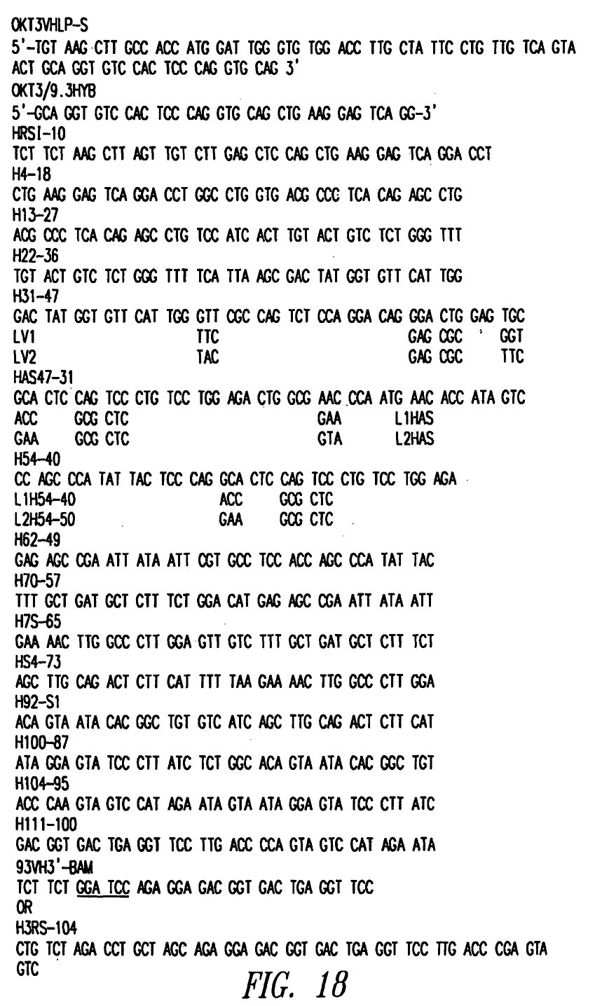

Llamalized 9.3 V_{H} molecules LV1 and LV2 were constructed as described for rederivation of the 9.3 V_{H}, using the oligo pairs with alterations in the sequence at residues 37, 44, 45, and 47 in the mature V_{H} (FIG. 18). These PCR products were digested with HindIII and BamHI and subcloned into the pXD expression vector. The vector also contained a BamHI-XbaI fusion protein cassette encoding the llama IgG₂ constant region. Similar constructs were also made using the llama IgG₁ and IgG₃ constant domains. The fusion protein expression cassette was then transiently transfected into COS cells in serum free medium and the supernatants were harvested 48 hours later. Culture supernatants were concentrated ten fold using AMICON filtration units, and 100 μl incubated with 10⁶ Jurkat T cells for 2 hours on ice. Cells were spun at 1300 rpm for 5 minutes, supernatants aspirated, and resuspended in 100 μl staining buffer (PBS, 2% FBS) containing 1:40 FITC anti-llama (Kent Labs) or FITC-anti mouse reagent (Biosource International) for 1 hour on ice. Cells were spun again at 1300 rpm for 5 minutes, supernatants aspirated, and washed in 200 μl staining buffer. Final cell pellets were resuspended in 400 μl staining buffer and analyzed with a FACSCAN cell sorter.

10.2 Results

Based on the observed characteristics of llama V_{HH} domain, a method was developed to convert non-llama antibody heavy chains to ones that would not require pairing with a light chain in a process herein referred to as llamalization. V_{H} sequences from isolated mAbs were determined or identified using sequence data available from the Genbank DNA sequence database. These sequences were used to design short, overlapping oligonucleotides encoding short peptides of the V_{H} domain. An accompanying PCR cycling method was developed which permitted de novo synthesis of the V_{H} domain using the appropriate combinations of these oligonucleotides. Sequence changes were incorporated into the oligonucleotides which spanned the residues identified as important in llama V_{HH} structural stability—11, 37, 44, 45, and 47 (Table II). In that regard, position 11 of any antibody may be changed to S, K, V, T or E; position 37 may be changed to Y, F, L, V, A or I; position 44 may be changed to E, D, K, T, Q, P, A or L; position 45 may be changed to R, L or I; and position 47 may be changed to F, G S, A, L, I, R, Y, M or W.

The llamalized V_{H} domains were subcloned as HindIII+XbaI fragments into pUC19 for sequence analysis. Once the sequence changes were verified, the cassettes were shuttled into a mammalian expression vector encoding a leader peptide and an Ig fusion domain for expression studies. Culture supernatants from transient transfection experiments were then screened for expression of soluble Ig fusion protein and antigen binding capacity.

The aforementioned method was applied to an anti-CD28 antibody 9.3 using the overlapping oligonucleotides shown in FIG. 18. A pair of complementary oligonucleotides were designed at the approximate midpoint of the antibody V-region coding sequence. The DNA duplex formed by these annealed oligonucleotides was the starting point for constructing the rest of the V-region using overlapping single stranded primers which extended the length of the starting oligonucleotides by 24 bases at both ends. Since the DNA was very short at this stage, cycling times during the PCR were kept very short and the molar amount of overlapping primer was kept low as well. With each consecutive amplification step, the molar concentration of newly added primer was increased and the cycling times were adjusted for slightly longer extensions. In this way, the de novo construction of the desired DNA sequence proceeded bidirectionally and was terminated by a final PCR that added unique restriction sites to each end of the DNA to facilitate cloning.

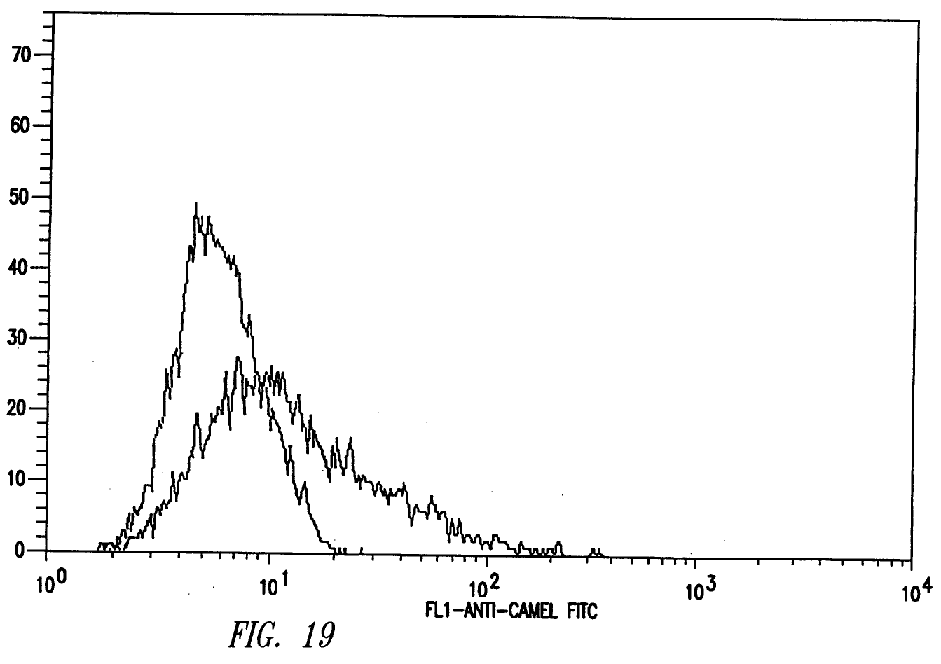

FIG. 19 shows a histogram display for Jurkat cells stained with llamalized version 2 of 9.3 antibody culture supernatant (10×) as compared with second step FITC-conjugated anti-llama antibody alone. The results demonstrate that a llamalized mouse anti-CD28 antibody was able to bind to its target antigen on cells as a heavy chain-only antibody.

11. Example: CDR Peptides Derived from Anti-Cd3 and Anti-Cd28 Antibodies Bound Target Antigens

This section describes the generation of soluble recombinant fusion proteins containing the extracellular domains of CD3δ, ε or γ subunit. Co-expression of CD3ε with either CD3γ or CD3δ results in fusion proteins that interacted at high affinities with a number of anti-CD3 mAbs including the ones that bound only to native conformational epitopes. Thus, this represents a method for producing native CD3ε/δ or CD3ε/γ heterodimers. This system is suitable for defining the conditions required for CD3 heterodimer formation, providing the tools to identify potential ligands for CD3 heterodimers, screening for molecules potentially capable of interfering with the interaction between the CD3 complex and the TCR on T cells.

11.1. Materials and Methods

11.1.1. Peptide Synthesis

Peptides corresponding to the entire CDR3 regions of anti-CD3 and anti-CD298 mAbs were synthesized, and Tyr/Phe-Cysteine residues were added to both amino and carboxyl termini. Modifications of peptides were made by eliminating one amino acid of the CDR3 region at a time from the terminus. Peptide synthesis was carried out on solid phase by using Fmoc chemistry (HBTU/DIEA activation and TFA cleavage). Crude peptides were combined in a batch of 3-5 peptides and cyclized by air oxidation at pH 8.5. Crude cyclic peptides were purified on a reverse phase HPLC, lyophilized and characterized by analytical HPLC and mass spectroscopy.

11.1.2. BIACORE

BIACORE uses surface plasmon resonance (SPR) which occurs when surface plasmon waves are excited at a metal/liquid interface. Light is directed at, and reflected due to bimolecular interactions between analyte (in solution) and ligand (immobilized). CD3εδhuIg, CD3εεhuIg and CD28huIg were covalently immobilized on a carboxymethy dextran chip using EDC/NHS chemistry followed by blocking with ethanol amine. Peptides were dissolved in HBS buffer at pH 7.2 with or without 1% DMSO, and were allowed to pass over these fusion protein-immobilized surfaces. Non-specific binding was substrated by passing these peptides over a controlled surface prepared by immobilizing EDC/NHS alone followed by ethanolamine.

11.1.3. Construction of CD3 Dimers

To generate a CD3ε-Ig fusion construct (phCD3ε-Ig), a cDNA encoding the extracellular domain of CD3ε including the native start codon and the leader sequence was amplified from total RNA of anti-CD3 plus anti-CD28-activated T cells (72 hours) by RT-PCR using the following primers set: Forward primer, 5′ GCG [CTC GAG] CCC ACC ATG CAG TCG GGC ACT CAC TGG (SEQ ID NO:55) and reverse primer 5′ GGC C[GG ATC C]GG ATC CAT CTC CAT GCA GTT CTC ACA (SEQ ID NO:56). Nucleotides in parenthesis are the XhoI (CTC GAG) and BamHI (GGA TCC) sites designed for cloning. PCR products were digested with XhoI and BamHI. The cut fragment was purified. A CDM8 expression vector harboring a genomic fragment encoding human IgG₁ hinge-CH2-CH3 was cut with XhoI and BamHI. Ligation of the cut vector and PCR product placed the cDNA encoding CD3ε extracellular domain in front of and in-frame with the genomic fragment encoding IgG₁ hinge-CH2-CH3. The CMV promoter in this vector controlled expression of CD3-Ig fusion protein in mammalian cells.

A cDNA fragment encoding human IgG₁ hinge-CH2-CH3 was used as a fusion partner for the CD3δ-(phCD3δ0Ig) and CD3γ-Ig (phCD3γ-Ig) constructs instead of a genomic fragment. This fragment was cloned into the BamHI and XbaI sites of the pD18 expression vector, also containing a CMV promoter for protein expression. Fragments of cDNA encoding the extracellular domains of CD3δ and CD3γ including the native start codons and leader sequences were isolated by RT-PCR from the same total RNA described above. The primers used are as follows:

| | |
|---|---|
| CD3δ forward, | |
| 5′ GCG ATA [AAG CTT] GCC ACC ATG GAA | (SEQ ID NO:57) |
| CAT AGC ACG TTT CTC, | |
| CD3δ reverse, | |
| 5′ GCG [GGA TCC] ATC CAG CTC CAC ACA | (SEQ ID NO:58) |
| GCT CTG, | |
| CD3γ forward | |
| 5′ GCG ATA [AAG CTT] GCC ACC ATG GAA | (SEQ ID NO:59) |
| CAG GGG AAG GGC CTG | |
| CD3γ reverse, | |
| 5′ GCG [GGA TCC] ATT TAG TTC AAT GCA | (SEQ ID NO:60) |
| GTT CTG AGA C. | |

Nucleotides in parenthesis are the HindIII (AAG CTT) and BamHI (GAA TTC) sites for cloning. PCR products were cut with HindIII and BamHI. Purified cut PCR fragments were then cloned into HindIII and BamHI cut hinge-CH2-CH3 containing pD18 vector. The cDNA encoding CD3δ and CD3γ extracellular domains was placed in front of and in-frame with that encoding IgG₁ hinge-CH2-CD3.

Because of the presence of two cysteine residues in the hinge region of the IgG₁ hinge-CH2-CH3 fragment that could form disulfide linkages, fusion proteins were usually expressed as dimers.

Transient expression in COS-7 cells was used to generate different CD3-Ig fusion proteins. The plasmids phCD3ε-Ig, and phCD3γ-Ig were transfected individually or in combinations of phCD3ε-Ig+phCD3δ-Ig and phCD3ε-Ig+phCD3γ-Ig into COS-7 by the DEAE-dextran method. Transfected cells were maintained in medium supplemented with a low concentration, 0.5%, FBS and insulin. Spent media were collected in three-day intervals up to three weeks post transfection. Fusion proteins were then purified from spent media by protein A-Sepharose chromatography. Fusion protein expression was confirmed by SDS-PAGE and ELISA using anti-CD3 mAb.

11.2. Results

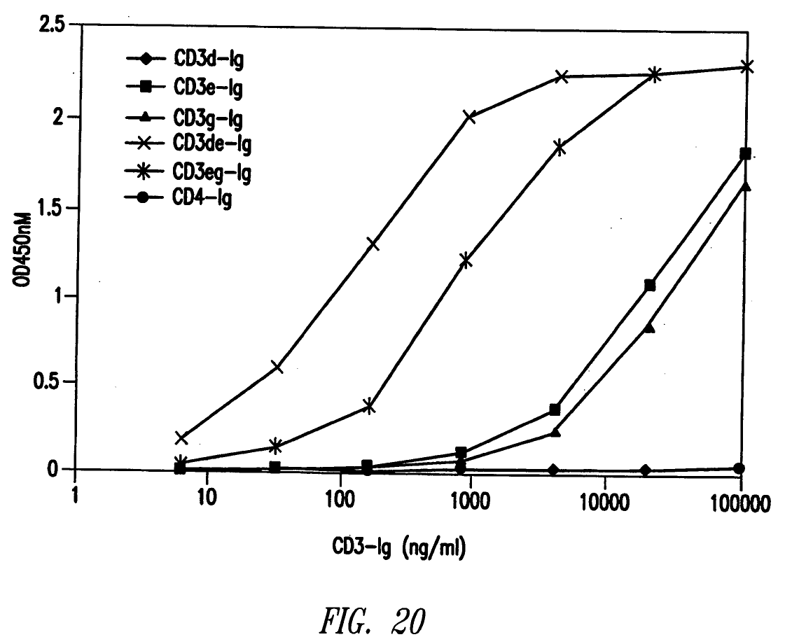

CD3-Ig fusion proteins were characterized by ELISA using a number of anti-CD3 mAbs including G19-4, OKT3, BC3, and 64.1 Anti-CD3 mAbs were immobilized to capture CD3-Ig. An antibody-horseradish peroxidase conjugate specific against human IgG hinge-CD2-CD3 was used to detect the binding of CD3-Ig to anti-CD3 mAbs. Like the control CD4-Ig, no binding of CD3δ-Ig to G19-4 was detectable even at 100 μg/ml of the fusion protein (FIG. 20). Although binding of CD3ε-Ig and CD3γ-Ig to G19-4 was detectable, it did not reach saturation even at concentrations as high as 100 μg/ml. On the other hand the CD3εδ-Ig and CD3εγ-Ig heterodimers bound to G19-4 at much higher affinities (FIG. 20). CD3εδ-Ig and CD3εγ-Ig saturated at 4 μg/ml and 20 μg/ml in this assay, respectively. Similarly, OKT3, BC3, and 64.1 anti-CD3 mAbs also showed much better binding to the CD3εδ-Ig heterodimer than the CD3εγ-Ig. These data indicate that co-expression of either CD3ε-Ig with CD3δ-Ig, or to some extent CD3ε-Ig with CD3γ-Ig, in COS cells resulted in heterodimeric CD3-Ig fusion proteins that were folded to their native conformation as defined by anti-CD3 mAbs. In addition, binding affinities of the CD3-Ig fusion proteins to different anti-CD3 antibodies were measured by BIACORE, and the results are shown in Table IV. Thus, CD3εδ and CD3εγ heterodimers may be used in detecting anti-CD3 antibody activity in antibody-coated plates or beads, as well as in screening of small molecules or peptides that bind specifically to CD3.

TABLE IV
Binding Affinities of Anti-CD3 Antibodies to CD3-Ig
Fusion Proteins As Measured By BIACORE

Affinity (nM)

| Anti-CD3 Antibody | CD3εδ-Ig | CD3εε-Ig |
|---|---|---|
| G19.4 | 1.28 | μM* |
| OKT-3 | 10.6 | μM* |
| BC-3 | 5.7 | μM* |
| 64.1 | 7.58 | μM* |
| MOPC (control) | Not detectable | Not detectable |

μM* = Binding was at micromolar level or below.

The CDR3 region of an anti-CD3 mAb and an anti-CD28 mAb was determined, and peptides corresponding to this region were synthesized. Cysteine residues were added to the ends of the peptides, followed by an aromatic residue tyrosine or tryptophan (Greene, WO95/34312). Upon air oxidation, the peptides were cyclized due to the formation of a disulphide linkage between the cysteines. As a result, the aromatic residues were in the exocyclic portion of the cyclized CDR peptides.

The binding affinities of the various peptides to their target antigens in the form of Ig fusion proteins were measured by BIACORE. Table V shows that a number of peptides exhibited high binding affinities for CD3εδ-Ig, whereas several peptides exhibited binding affinities for CD28-Ig. Thus, small CDR peptides may be used in lymphocyte activation in place of antibodies.

TABLE V

Binding Affinities of Peptides Derived From
CDR3 Regions Of Two mAbs

| | Binding Affinity | |
|---|---|---|
| Peptides* | CD3εδIg** | CD28Ig |

| | | | | | |
|---|---|---|---|---|---|
| YCRSAYYDYDGIAYCW | (SEQ ID NO:61) | 7 | μM | 166 | μM |
| YCSAYYDYDGIAYCW | (SEQ ID NO:62) | | | | |
| YCAYYDYDGIAYCW | (SEQ ID NO:63) | | | | |

| | | | |
|---|---|---|---|
| YCRYYDDHYSLDYCW | (SEQ ID NO:64) | nd | nd |
| YCYYDDHYSLDYCW | (SEQ ID NO:65) | | |
| YCYDDHYSLDYCW | (SEQ ID NO:66) | | |
| YCDDHYSLDYCW | (SEQ ID NO:67) | | |
| YCDHYSLDYCW | (SEQ ID NO:68) | | |

| | | | | |
|---|---|---|---|---|
| YCARDSDWYFDVCW | (SEQ ID NO:69) | 50 | μM | nd |
| YCARSDWYFDVCW | (SEQ ID NO:70) | | | |
| YCARDWYFDVCW | (SEQ ID NO:71) | | | |

| | | | | |
|---|---|---|---|---|
| YCGYSYYYSMDYCW | (SEQ ID NO:72) | nd | 1.0 | μM |
| YCYSYYYSMDYCW | (SEQ ID NO:73) | | | |
| YCSYYYSMDYCW | (SEQ ID NO:74) | | | |

| | | | | |
|---|---|---|---|---|
| YCYDYDGCY | (SEQ ID NO:75) | 10 | μM | nd |

| | | | |
|---|---|---|---|
| YCYDYDYCY | (SEQ ID NO:76) | nd | nd |
| YCYDYDFCY | (SEQ ID NO:77) | nd | nd |
| YCYDDHTCY | (SEQ ID NO:78) | nd | nd |
| YCYDDHQCY | (SEQ ID NO:79) | nd | nd |

| | | | | |
|---|---|---|---|---|
| YCFDWKNCY | (SEQ ID NO:80) | 0.5 | μM | nd |

* = Peptides were made individually, pooled in a batch of 3-5 peptides, cyclized, purified and characterized as pools.
** = CD3εδhuIg used for the binding affinity was impure and was a mixture of several components which were not fully characterized.
nd = non detectable binding

The present invention is not to be limited in scope by the exemplified embodiments which are intended as illustrations of single aspects of the invention and any sequences which are functionally equivalent are within the scope of the invention. Indeed, various modifications of the invention in addition to those shown and described herein will become apparent to those skilled in the art from the foregoing description and accompanying drawings. Such modifications are intended to fall within the scope of the appended claims.

All publications cited herein are incorporated by reference in their entirety.

1. A method for activating a lymphocyte, comprising aggregating three or more antigens expressed by the lymphocyte, thereby activating the lymphocyte.

2. The method of claim 1 in which the lymphocyte is a T cell.

3. The method of claim 2 in which the T cell expresses CD4.

4. The method of claim 2 in which the three or more antigens are selected from a combination of CD2, CD3, CD4, CD5, CD6, CD8, CD18, CD25, CD27, CD28, CD40, CD43, CD45, CD45RA, CD45RO, CDw137, CDW150, CD152, CD154, ICOS, TCR alpha, TCR beta, TCR delta, TCR gamma, and a cytokine receptor.

5. (Canceled)

6. The method of claim 4 in which the antigens are aggregated by one or more antibodies or an antigen-binding derivative thereof.

7. The method of claim 6 in which the antibody contains only heavy chains or an antigen-binding derivative thereof.

8. The method of claim 7 in which the antigen-binding derivatives are V_{HH}.

9. (Canceled)

10. (Canceled)

11. The method of claim 4 in which the antigens are aggregated by their corresponding ligands.

12. The method of claim 6 in which the antibodies or antigen-binding derivatives are immobilized on a solid surface.

13. The method of claim 12 in which the antibodies or antigen-binding derivatives are conjugated to a particulate substrate.

14. The method of claim 12 in which the antibodies or antigen-binding derivatives are arranged in a sequential order.

15. The method of claim 2 in which the T cell is activated to proliferate.

16. The method of claim 2 in which the T cell is activated to produce cytokines.

17. The method of claim 2 in which the T cell is activated to alter its expression of cell surface antigens.

18. The method of claim 2 in which the T cell is activated to alter its expression of cytokines.

19. The method of claim 2 in which the T cell is activated to undergo apoptosis.

20.-49. (Canceled)

50. The method of claim 2 wherein the three or more antigens comprise at least CD3 and CD28.

51. The method of claim 50 wherein the three or more antigens further comprise CD2.

52. The method of claim 50 wherein the three or more antigens further comprise CD18.

53. The method of claim 50 wherein the three or more antigens further comprise CD40.

54. The method of claim 2 wherein the three or more antigens comprise CD28 and TCRVβ.


SEQUENCE LISTING (80 sequences)

SEQ ID NO: 1 - protein, 225 aa, Llama llama
IRLLVESGGGLAQPGGSLRLSCAASGVQEGLDGMGWYRQAPGKQPELVAGISSTNIPNYSKSVKGRFTISRDNAKNTVYLQMNSLKPEDTAVYYCNADKRGPVITVYWGKGTQVTVSSEPKTPKPQPQPQPQPQPNPTTESKCPKCPAPELLGGPSVFIFPPKPKDVLSISGRPEVTCVVVDVGQEDPEVSFNGTLMARGVWRGLVQPGGSLTLSVNLDLLRLYS

SEQ ID NO: 2 - protein, 183 aa, Llama llama
IRLLVESGGGLVRAGGSLRLSCAASGRIFSNYTLGWFRQAPGKEPEFVADISGSITFYADSVKGRFTISRDNAQNTVYLQMNLLKFADTAVYYCAASEDRRTELKKERANSWFPARKFMQYEYWGQGTQVAVSSEPKTPKPQPQPQPQPQPNPTTESKCPKCPAPELLGGPSVLSSPPKPKDV

SEQ ID NO: 3 - protein, 204 aa, Llama llama
IRLLVESGGGLVQAGGSLRLSCVASGRIFTIRTMGWYRQTPGIQPELVAEITADGSQNYVDSVKGRFTIFGDNDKKTVWLQMNSLKAEDTADYYCAADIITTDWRSSRYWGQGTQVTVSSEPKTPKPQPQPQPQPQPNPTTESKCPKCPAPELLGGPSVFIFPPKPKDVLSISGRPEVTCVVVDVGQEDPEVSFNGTLMAKAEF

SEQ ID NO: 4 - protein, 208 aa, Llama llama
IRLLVESGGGLVQPGGSLRLSCAASERDFGSSVMGWFRQAPGKEPEFVAAINWSVGGTYYTDSVKGRFTISRDNAKNTVYLQMNSLKPEDTAVYSCAVRTRQRLNIRADEDYGYWGQGTQVTVSSEPKTPKPQPQPQPQPQPNPTTESKCPKCPAPELLGGPSVFIFPPKPKDVLSISGRPEVTCVVVDVGQEDPEVSFNGTLMAKPN

SEQ ID NO: 5 - protein, 206 aa, Llama llama
IRLLVESGGGLVQAGGSLRLSCTTSGIKFGITAMTWYRQTPLNEPELVAVVGGGGSTLYEGRVKGRFTISRDNDKNTAYLQMNSLKPEDTAVYYCGAAASILAASSAETVQYWGQGTQVTVSLEPKTPKPQPQPQPQPQPNPTTESKCPKCPAPELLGGPSVFIFPPKPKDVLSISGRPEVTCVVVDVGQEDPEVSFNGTLMAKPN

SEQ ID NO: 6 - protein, 208 aa, Llama llama
IRLLVESGGGLVQRGASLRLTCVVSGIFVDRWAMGWFRQAPGQKPLFVASIAWDGDETWYGDSVKGRFTVSRDVAKNSVYLQLANLQPEDTATYSCAALNGAWPSSIATMTPDLGWWGQGTQVTVSLEPKTPKPQPQPQPQPNPTTESKCPKCPAPELLGGPSVFIFPPKPKDVLSISGRPEVTCVVVDVGQEDPEVSFNGTLMAKPN

SEQ ID NO: 7 - protein, 204 aa, Llama llama
IRLLVESGGGLVQTGDSLKLSCVASGRNFSSYHMAWFRQTPDKEPEFVAVSWKGGSEYYKNSVKGRFTLSRDGAKNTVYLQMNSLKPEDSGVYYCAADDHVTRGASKASYRYWGQGTQVTVSSEPKTPKPQPQPQPQPNPTTESKCPKCPAPELLGGPSVFIFPPKPKDVLSISGRPEVTCVVVDVGQEDPEVSFNGTLMAKPN

SEQ ID NO: 8 - protein, 211 aa, Llama llama
IRLLVESGGGLVQAGGSLRLSCTASGRTFSRYYMGWFRQAPGKEPESVALISRSGGSTDYADSVKGRFTISRDNAKNTPYLQMNSLIPEDTADYYCAANIAAGWDTLSRDWRDKRTYSYWGQGTQVTVSSEPKTPKPQPQPQPQPNPTTESKCPKCPAPELLGGPSVFIFPPKPKDVLSISGRPEVTCVVVDVGQEDPEVSFNGTLMAKPN

SEQ ID NO: 9 - protein, 205 aa, Llama llama
IRLLVESGGGLVQAGDSLRLSCAASGRTFTNYAMGWFRQAPGKEPEFVARISRVGSSTFYTDSVKGRFTISRDNAKNTMYLQMNSMKAEDTAVYYCAADSDYGPGRRSSEYDYWGQGTQVTVSSEPKTPKPQPQPQPQPNPTTESKCPKRPAPELLGGPSVFIFPPKPKDVLSISGRPEVTCVVVDVGQEDPEVSFNGTLMAKPN

SEQ ID NO: 10 - protein, 209 aa, Llama llama
IRLLVESGGGLVQAGGSLQLSCATSGVLTSGDYAVGWFRQAPGKEREGVSCLSRYGGPTLYADSVKGRFTSSSDAAKTKVYLQMNNLKPEDTAVYYCTAHISCDWNIINPNEYDYWGQGTQVTVSSEPKTPKPQPQPQPQPQPNPTTESKCPKCPAPELLGGPSVFIFPPKPKDVLSISGRPEVTCVVVDVGQEDPEVSFNGTLMASRI

SEQ ID NO: 11 - protein, 217 aa, Llama llama
IRLLVESGGGLVQPGDSLRLSCAVSGVFTLDDYAIGWFRQAPGKEREGVICMSASDGSTYYSDSVKGRFTISRDDDKNTLYLQMERLKPEDTATYYCAANYLGRVRGSAIRAADYCSGSGSVVYHFWGQGTQVTVSSEPKTPKPQPQPQPQPNPTTESKCPKCPAPELLGGPSVFIFPPKPKDVLSISGRPEVTCVVVDVGQEDPEVSFNGTLMAEF

SEQ ID NO: 12 - protein, 219 aa, Llama llama
IRLLVESGGGLVQPGGSLRLSCAASGVFTRDYYVIAWFRQAPGKEREGVSCISTRGSTYYADSVKGRFAISGDNDKMTVYLQMNNLKPEDTAVYYCGALINWYSPPNTDYDSAWCRGRSLGDYGLDYWGKGTLVTVSSEPKTPKPQPQPQPQPNPTTESKCPKCPAPELLGGPSVFIFPPKPKDVLSISGRPEVTCVVVDVGQEDPEVSFNGTLMAKPN

SEQ ID NO: 13 - protein, 216 aa, Llama llama
IRLLVESGGGLVQAGGSLRLSCAASGVFTFDDYAIAWFRQAPGKEREGVSCISTSDGSTYYGGSVKGRFTISVDVAKNTVYLQMNSLKPDDTAVYYCAADPRIWLHSVVQGTERCLTNDYDYWGQGTQVTVSSELKTPKPQPQPQPQPQLNPTTESKCPKCPAPELLGGPSVFIFPPKPKDVLSISGRPEVTCVVVDVGQEDPEVSFNGTLMAKPN

SEQ ID NO: 14 - protein, 214 aa, Llama llama
IRLLVESGGGLVQPGGSLTLSCETFGVSTSDYYYIGWIRQAPGRERERVSCISGRDGTAAYADSVKGRFTISRDNAKNTVYLQMNNLKPEDTADYYCTANLGLRPSDFNRGYKCPYEYDYWGQGTQVTVSSEPKTPKPQPQPQPQPQPNPTTESKCPKCPAPELLGGPSVFIFPPKPKDVLSISGRPEVTCVVVDVGQEDPEVSFNGTLMASRI

SEQ ID NO: 15 - protein, 204 aa, Llama llama
IRLLVESGGGLVQAGGSLRLSCAASGVLTFDDYDIGWFRQAPEKDREGVSCISATDNTTYYSDSVKGRFTISSNNAENTVYLQINSLQPEDTAVYHCAAVRSWVKSIYSRTWCTDLYLEVWGQGTLVTVSSEPKTPKPQPQPQPQPLPNPTTESKCPKCPAPELLGGPSVFIFPPKPKDVLSISGRPEVTCVVVDVGQEDPSRI

SEQ ID NO: 16 - protein, 231 aa, Llama llama
EPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSRLSVGKNTWQRGETLTCVVMHEALHNHYTQKSITQSSGK

SEQ ID NO: 17 - protein, 231 aa, Llama llama
EPHGGCTCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPVPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSITQSSGK

SEQ ID NO: 18 - protein, 246 aa, Llama llama
EPKTPKPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYTLAPHREELAKDTVSVTCLVKGFYPPDINVEWQRNGQPESEGTYATTPPQLDNDGTYFLYSKLSVGKNTWQQGETFTCVVMHEALHNHYTQKSITQSSGK

SEQ ID NO: 19 - protein, 248 aa, Llama llama
EPKTPKPQPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYTLAPHREELAKDTVSVTCLVKGFYPPDINVEWQRNGQPESEGTYATTPPQLDNDGTYFLYSKLSVGKNTWQQGETFTCVVMHEALHNHYTQKSITQSSGK

SEQ ID NO: 20 - protein, 250 aa, Llama llama
EPKTPKPQPQPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYTLAPHREELAKDTVSVTCLVKGFYPPDINVEWQRNGQPESEGTYATTPPQLDNDGTYFLYSKLSVGKNTWQQGETFTCVVMHEALHNHYTQKSITQSSGK

SEQ ID NO: 21 - protein, 234 aa, Llama llama
AHHSEDPTSKCPKCPGPELLGGPTVFIFPPKAKDVLSITRKPEVTCVVVDVGKEDPEINFSWSVDGTEVHTAETKPKEEQLNSTYRVVSVLPIQHQDWLTGKEFKCKVNNKALPAPIERTISKAKGQTREPQVYTLAPHREELAKDTVSVTCLVKGFFPADINVEWQRNGQPESEGTYANTPPQLDNDGTYFLYSKLSVGKNTWQQGEVFTCVVMHEALHNHSTQKSITQSSGK

SEQ ID NO: 22 - DNA, 81 bp, Artificial Sequence
  Primer
tgtaagcttgccaccatggattgggtgtggaccttgctattcctgttgtcagtaactgcaggtgtccactcccaggtgcag

SEQ ID NO: 23 - DNA, 38 bp, Artificial Sequence
  Artificially synthesized sequence
gcaggtgtccactcccaggtgcagctgaaggagtcagg

SEQ ID NO: 24 - DNA, 48 bp, Artificial Sequence
  Artificially synthesized sequence
tcttctaagcttagttgtcttgagctccagctgaaggagtcaggacct

SEQ ID NO: 25 - DNA, 45 bp, Artificial Sequence
  Artificially synthesized sequence
ctgaaggagtcaggacctggcctggtgacgccctcacagagcctg

SEQ ID NO: 26 - DNA, 45 bp, Artificial Sequence
  Artificially synthesized sequence
acgccctcacagagcctgtccatcacttgtactgtctctgggttt

SEQ ID NO: 27 - DNA, 45 bp, Artificial Sequence
  Artificially synthesized sequence
tgtactgtctctgggttttcattaagcgactatggtgttcattgg

SEQ ID NO: 28 - DNA, 51 bp, Artificial Sequence
  Artificially synthesized sequence
gactatggtgttcattgggttcgccagtctccaggacagggactggagtgc

SEQ ID NO: 29 - DNA, 51 bp, Artificial Sequence
  Artificially synthesized sequence
gactatggtgttcattggttccgccagtctccaggacaggagcgcgagggt

SEQ ID NO: 30 - DNA, 51 bp, Artificial Sequence
  Artificially synthesized sequence
gactatggtgttcattggtaccgccagtctccaggacaggagcgcgagttc

SEQ ID NO: 31 - DNA, 51 bp, Artificial Sequence
  Artificially synthesized sequence
gcactccagtccctgtcctggagactggcgaacccaatgaacaccatagtc

SEQ ID NO: 32 - DNA, 51 bp, Artificial Sequence
  Artificially synthesized sequence
accctcgcgctcctgtcctggagactggcggaaccaatgaacaccatagtc

SEQ ID NO: 33 - DNA, 51 bp, Artificial Sequence
  Artificially synthesized sequence
gaactcgcgctcctgtcctggagactggcggtaccaatgaacaccatagtc

SEQ ID NO: 34 - DNA, 44 bp, Artificial Sequence
  Artificially synthesized sequence
ccagcccatattactcccaggcactccagtccctgtcctggaga

SEQ ID NO: 35 - DNA, 44 bp, Artificial Sequence
  Artificially synthesized sequence
ccagcccatattactcccagaccctcgcgctcctgtcctggaga

SEQ ID NO: 36 - DNA, 44 bp, Artificial Sequence
  Artificially synthesized sequence
ccagcccatattactcccaggaactcgcgctcctgtcctggaga

SEQ ID NO: 37 - DNA, 42 bp, Artificial Sequence
  Artificially synthesized sequence
gagagccgaattataattcgtgcctccaccagcccatattac

SEQ ID NO: 38 - DNA, 42 bp, Artificial Sequence
  Artificially synthesized sequence
tttgctgatgctctttctggacatgagagccgaattataatt

SEQ ID NO: 39 - DNA, 42 bp, Artificial Sequence
  Artificially synthesized sequence
gaaaacttggcccttggagttgtctttgctgatgctctttct

SEQ ID NO: 40 - DNA, 42 bp, Artificial Sequence
  Artificially synthesized sequence
agcttgcagactcttcatttttaagaaaacttggcccttgga

SEQ ID NO: 41 - DNA, 42 bp, Artificial Sequence
  Artificially synthesized sequence
acagtaatacacggctgtgtcatcagcttgcagactcttcat

SEQ ID NO: 42 - DNA, 42 bp, Artificial Sequence
  Artificially synthesized sequence
ataggagtatcccttatctctggcacagtaatacacggctgt

SEQ ID NO: 43 - DNA, 42 bp, Artificial Sequence
  Artificially synthesized sequence
accccagtagtccatagaatagtaataggagtatcccttatc

SEQ ID NO: 44 - DNA, 42 bp, Artificial Sequence
  Artificially synthesized sequence
gacggtgactgaggttccttgaccccagtagtccatagaata

SEQ ID NO: 45 - DNA, 36 bp, Artificial Sequence
  Artificially synthesized sequence
tcttctggatccagaggagacggtgactgaggttcc

SEQ ID NO: 46 - DNA, 57 bp, Artificial Sequence
  Artificially synthesized sequence
ctgtctagacctgctagcagaggagacggtgactgaggttccttgacccgagtagtc

SEQ ID NO: 47 - DNA, 20 bp, Artificial Sequence
  Primer
ctcgtggartctggaggagg

SEQ ID NO: 48 - DNA, 44 bp, Artificial Sequence
  Primer
cgtcatgtcgacggatccaagctttgaggagacggtgacytggg

SEQ ID NO: 49 - DNA, 23 bp, Artificial Sequence
  Primer
caggtgcagctggtgcagtctgg

SEQ ID NO: 50 - DNA, 21 bp, Artificial Sequence
  Primer
ggttgtggttttggtgtcttg

SEQ ID NO: 51 - DNA, 24 bp, Artificial Sequence
  Primer
caggtcaactrraagggagtctgg

SEQ ID NO: 52 - DNA, 22 bp, Artificial Sequence
  Primer
caggtgcagctgcaggagtcgg

SEQ ID NO: 53 - DNA, 23 bp, Artificial Sequence
  Primer
taatacgactcactatagggaga

SEQ ID NO: 54 - DNA, 16 bp, Artificial Sequence
  Primer
aacagctatgaccatg

SEQ ID NO: 55 - DNA, 36 bp, Artificial Sequence
  Primer
gcgctcgagcccaccatgcagtcgggcactcactgg

SEQ ID NO: 56 - DNA, 36 bp, Artificial Sequence
  Primer
ggccggatccggatccatctccatgcagttctcaca

SEQ ID NO: 57 - DNA, 38 bp, Artificial Sequence
  Primer
gcgataaagctgccaccatggaacatagcacgtttctc

SEQ ID NO: 58 - DNA, 30 bp, Artificial Sequence
  Primer
gcgggatccatccagctccacacagctctg

SEQ ID NO: 59 - DNA, 39 bp, Artificial Sequence
  Primer
gcgataaagcttgccaccatggaacaggggaagggcctg

SEQ ID NO: 60 - DNA, 34 bp, Artificial Sequence
  Primer
gcgggatccatttagttcaatgcagttctgagac

SEQ ID NO: 61 - protein, 16 aa, Mus musculus
YCRSAYYDYDGIAYCW

SEQ ID NO: 62 - protein, 15 aa, Mus musculus
YCSAYYDYDGIAYCW

SEQ ID NO: 63 - protein, 14 aa, Mus musculus
YCAYYDYDGIAYCW

SEQ ID NO: 64 - protein, 15 aa, Mus musculus
YCRYYDDHYSLDYCW

SEQ ID NO: 65 - protein, 14 aa, Mus musculus
YCYYDDHYSLDYCW

SEQ ID NO: 66 - protein, 13 aa, Mus musculus
YCYDDHYSLDYCW

SEQ ID NO: 67 - protein, 12 aa, Mus musculus
YCDDHYSLDYCW

SEQ ID NO: 68 - protein, 11 aa, Mus musculus
YCDHYSLDYCW

SEQ ID NO: 69 - protein, 14 aa, Mus musculus
YCARDSDWYFDVCW

SEQ ID NO: 70 - protein, 13 aa, Mus musculus
YCARSDWYFDVCW

SEQ ID NO: 71 - protein, 12 aa, Mus musculus
YCARDWYFDVCW

SEQ ID NO: 72 - protein, 14 aa, Mus musculus
YCGYSYYYSMDYCW

SEQ ID NO: 73 - protein, 13 aa, Mus musculus
YCYSYYYSMDYCW

SEQ ID NO: 74 - protein, 12 aa, Mus musculus
YCSYYYSMDYCW

SEQ ID NO: 75 - protein, 9 aa, Mus musculus
YCYDYDGCY

SEQ ID NO: 76 - protein, 9 aa, Mus musculus
YCYDYDYCY

SEQ ID NO: 77 - protein, 9 aa, Mus musculus
YCYDYDFCY

SEQ ID NO: 78 - protein, 9 aa, Mus musculus
YCYDDHTCY

SEQ ID NO: 79 - protein, 9 aa, Mus musculus
YCYDDHQCY

SEQ ID NO: 80 - protein, 9 aa, Mus musculus
YCFDWKNCY